(19) United States
(12) Patent Application Publication
(10) Pub. No.: US 2024/0216396 A1
(43) Pub. Date: Jul. 4, 2024

(54) NEUROACTIVE STEROID FOR USE IN TREATING MAJOR DEPRESSIVE DISORDER AND POSTPARTUM DEPRESSION IN A LACTATING FEMALE

(71) Applicant: Sage Therapeutics, Inc., Cambridge, MA (US)

(72) Inventors: Robert Alfonso Lasser, Brookline, MA (US); James Doherty, Bedford, MA (US); Jeffrey Martin Jonas, Vero Beach, FL (US); Stephen Jay Kanes, Villanova, PA (US); Handan Gunduz-Bruce, Lexington, MA (US); Amy E. Bullock, Brookline, MA (US); Jeffrey A. Wald, Cary, NC (US)

(21) Appl. No.: 18/557,705

(22) PCT Filed: Apr. 29, 2022
(86) PCT No.: PCT/US2022/026908

§ 371 Date: Oct. 27, 2023

Related U.S. Application Data
(60) Provisional application No. 63/181,807, filed on Apr. 29, 2021.

(51) Int. Cl. A61K 31/58; A61K 9/00; A61K 49/00

(52) U.S. Cl. CPC A61K 31/58; A61K 9/0053; A61K 49/0004

(57) ABSTRACT

The present disclosure relates to Compound (1), or a pharmaceutically acceptable salt thereof, for use in methods of treating postpartum depression (PPD) in a human female subject during the subject's postnatal period, wherein the subject breastfeeds a child during the treatment period. The disclosure also relates to Compound (1), or a pharmaceutically acceptable salt thereof, for use in methods of treating major depressive disorder (MDD) in a human female subject, wherein the subject breastfeeds a child during the treatment period.

### CROSS-REFERENCE TO RELATED APPLICATIONS

This application claims the benefit of U.S. Provisional Application No. 63/181,807, filed on Apr. 29, 2021. The entire contents of the aforementioned application are incorporated herein by reference in their entireties.

### FIELD OF THE INVENTION

The present disclosure is directed to methods of treating postpartum depression (PPD) in a human female subject during the subject's postnatal period, the method comprising administering to the subject a therapeutically effective amount of Compound (1), or a pharmaceutically acceptable salt thereof, for a treatment period, wherein the subject breastfeeds a child during the treatment period. The disclosure is also directed to methods of treating major depressive disorder (MDD) in a human female subject, the method comprising administering to the subject a therapeutically effective amount of Compound (1), or a pharmaceutically acceptable salt thereof, for a treatment period, wherein the subject breastfeeds a child during the treatment period.

### BACKGROUND

Progesterone and its metabolites have been demonstrated to have profound effects on brain excitability (Backstrom, T. et al., Acta Obstet. Gynecol. Scand. Suppl. 130: 19-24 (1985); Pfaff, D. W and McEwen, B. S., Science 219:808-814 (1983); Gyermek et al, J Med Chem. 11: 117 (1968); Lambert, J. et al., Trends Pharmacol. Sci. 8:224-227 (1987)). The levels of progesterone and its metabolites vary with the phases of the menstrual cycle. It has been well documented that the levels of progesterone and its metabolites decrease prior to the onset of menses. The monthly recurrence of certain physical symptoms prior to the onset of menses has also been well documented. These symptoms, which have become associated with premenstrual syndrome (PMS), include stress, anxiety, and migraine headaches (Dalton, K., Premenstrual Syndrome and Progesterone Therapy, 2nd edition, Chicago Yearbook, Chicago (1984)). Subjects with PMS have a monthly recurrence of symptoms that are present in premenses and absent in postmenses.

A syndrome related to low progesterone levels is postnatal depression (PND) or postpartum depression (PPD). Immediately after birth, progesterone levels decrease dramatically leading to the onset of PND. The symptoms of PND range from mild depression to psychosis requiring hospitalization. PND is also associated with severe anxiety and irritability. PND-associated depression is not amenable to treatment by classic antidepressants, and women experiencing PND show an increased incidence of PMS (Dalton, K., Premenstrual Syndrome and Progesterone Therapy, 2nd edition, Chicago Yearbook, Chicago (1984)).

There is increasing evidence to support the use of neuroactive steroids, e.g., a neuroactive steroid as described herein, e.g., Compound (1); in the treatment and prevention of postpartum depression.

### SUMMARY OF THE INVENTION

In one aspect, the disclosure provides a method of treating postpartum depression (PPD) in a human female subject during the subject's postnatal period, the method comprising administering to the subject a therapeutically effective amount of Compound (1):

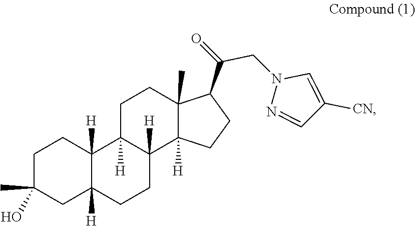

for a treatment period, wherein the subject breastfeeds a child during the treatment period.

In one aspect, the disclosure provides a method of treating postpartum depression (PPD) in a human female subject during the subject's postnatal period, the method comprising administering to the subject a therapeutically effective amount a pharmaceutically acceptable salt of Compound (1):

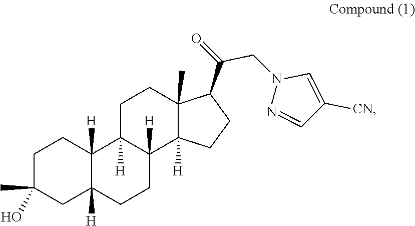

for a treatment period, wherein the subject breastfeeds a child during the treatment period.

In one aspect, the disclosure provides a method of treating postpartum depression (PPD) with elevated anxiety in a human female subject during the subject's postnatal period, the method comprising administering to the subject a therapeutically effective amount of Compound (1):

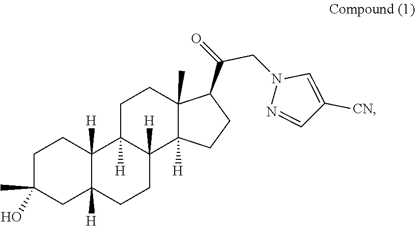

for a treatment period, wherein the subject breastfeeds a child during the treatment period.

In one aspect, the disclosure provides a method of treating postpartum depression (PPD) with elevated anxiety in a human female subject during the subject's postnatal period, the method comprising administering to the subject a therapeutically effective amount a pharmaceutically acceptable salt of Compound (1):

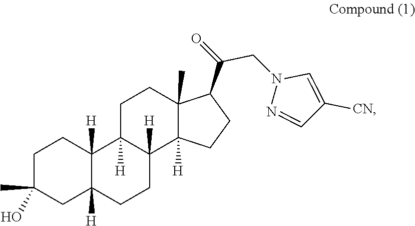

for a treatment period, wherein the subject breastfeeds a child during the treatment period.

In some embodiments, the subject breastfeeds the child at least 3 times per day.

In some embodiments, the treatment period is about 2 weeks or about 14 days.

In some embodiments, Compound (1), or the pharmaceutically acceptable salt of Compound (1), is administered once a day for about 14 days or about 2 weeks. In some embodiments, Compound (1) is administered at a dose of about 20 mg to about 55 mg. In some embodiments, Compound (1) is administered at a dose of about 50 mg. In some embodiments, Compound (1) is administered at a dose of about 40 mg. In some embodiments, Compound (1) is administered at a dose of about 30 mg.

In some embodiments, the pharmaceutically acceptable salt of Compound (1) is administered at a dose equivalent to about 20 mg to about 55 mg of the free base compound. In some embodiments, the pharmaceutically acceptable salt of Compound (1) is administered at a dose equivalent to about 50 mg of the free base compound. In some embodiments, the pharmaceutically acceptable salt of Compound (1) is administered at a dose equivalent to about 40 mg of the free base compound. In some embodiments, the pharmaceutically acceptable salt of Compound (1) is administered at a dose equivalent to about 30 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally. In some embodiments, Compound (1), or the pharmaceutically acceptable salt of Compound (1), is administered with food. In some embodiments, Compound (1), or the pharmaceutically acceptable salt of Compound (1), is administered once a day at night.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the subject is treatment naïve.

In some embodiments, the subject has been on a stable dose of an additional antidepressant for at least 30 days or for at least 60 days prior to the beginning of the treatment period.

In some embodiments, the breast milk of the subject is monitored to determine relative infant dose of Compound (1), or the pharmaceutically acceptable salt of Compound (1), in the breast milk, and the daily dose of Compound (1) is adjusted to produce less than a maximum relative infant dose. In some embodiments, the maximum relative infant dose is at most about 0.5% of the daily dose administered to the subject. In some embodiments, the maximum relative infant dose is at most about 0.4% of the daily dose administered to the subject. In some embodiments, the maximum relative infant dose is at most about 0.357% of the daily dose administered to the subject.

In some embodiments, the child is monitored for abnormal behavior. In some embodiments, the abnormal behavior is selected from the group consisting of agitation, irritability, lethargy, oversleeping, poor feeding, and poor weight gain.

In some embodiments, the daily dose administered to the subject is decreased by 10 mg if the relative infant dose is above the maximum relative infant dose, or if the child shows abnormal behavior.

Another aspect of the disclosure provides a method of treating major depressive disorder (MDD) in a human female subject, the method comprising administering to the subject a therapeutically effective amount of Compound (1):

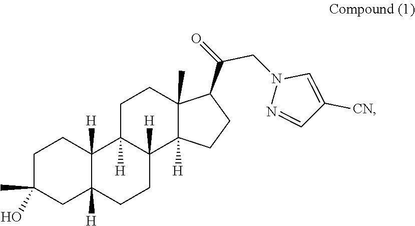

for a treatment period, wherein the subject breastfeeds a child during the treatment period.

In one aspect, the disclosure provides a method of treating major depressive disorder (MDD) in a human female subject, the method comprising administering to the subject a therapeutically effective amount a pharmaceutically acceptable salt of Compound (1):

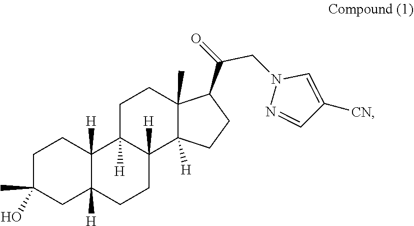

for a treatment period, wherein the subject breastfeeds a child during the treatment period.

In one aspect, the disclosure provides a method of treating major depressive disorder (MDD) with elevated anxiety in a human female subject, the method comprising administering to the subject a therapeutically effective amount of Compound (1):

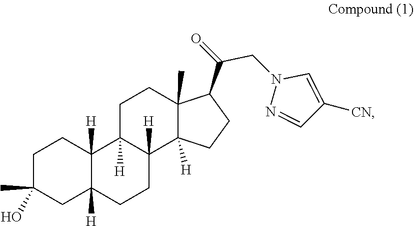

for a treatment period, wherein the subject breastfeeds a child during the treatment period.

In one aspect, the disclosure provides a method of treating major depressive disorder (MDD) with elevated anxiety in a human female subject, the method comprising administering to the subject a therapeutically effective amount a pharmaceutically acceptable salt of Compound (1):

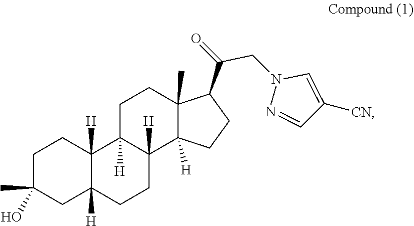

for a treatment period, wherein the subject breastfeeds a child during the treatment period.

In some embodiments, the subject breastfeeds the child at least 3 times per day.

In some embodiments, the treatment period is about 2 weeks or about 14 days.

In some embodiments, Compound (1), or the pharmaceutically acceptable salt of Compound (1), is administered once a day for about 14 days or about 2 weeks. In some embodiments, Compound (1) is administered at a dose of about 20 mg to about 55 mg. In some embodiments, Compound (1) is administered at a dose of about 50 mg. In some embodiments, Compound (1) is administered at a dose of about 40 mg. In some embodiments, Compound (1) is administered at a dose of about 30 mg.

In some embodiments, the pharmaceutically acceptable salt of Compound (1) is administered at a dose equivalent to about 20 mg to about 55 mg of the free base compound. In some embodiments, the pharmaceutically acceptable salt of Compound (1) is administered at a dose equivalent to about 50 mg of the free base compound. In some embodiments, the pharmaceutically acceptable salt of Compound (1) is administered at a dose equivalent about 40 mg of the free base compound. In some embodiments, the pharmaceutically acceptable salt of Compound (1) is administered at a dose equivalent about 30 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally. In some embodiments, Compound (1), or the pharmaceutically acceptable salt of Compound (1), is administered with food. In some embodiments, Compound (1), or the pharmaceutically acceptable salt of Compound (1), is administered once a day at night.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the subject is treatment naïve.

In some embodiments, the subject has been on a stable dose of an additional antidepressant for at least 30 days or for at least 60 days prior to the beginning of the treatment period.

In some embodiments, the breast milk of the subject is monitored to determine relative infant dose of Compound (1), or the pharmaceutically acceptable salt of Compound (1), in the breast milk, and the daily dose of Compound (1) is adjusted to produce less than a maximum relative infant dose. In some embodiments, the maximum relative infant dose is at most about 0.5% of the daily dose administered to the subject. In some embodiments, the maximum relative infant dose is at most about 0.4% of the daily dose administered to the subject. In some embodiments, the maximum relative infant dose is at most about 0.357% of the daily dose administered to the subject.

In some embodiments, the child is monitored for abnormal behavior. In some embodiments, the abnormal behavior is selected from the group consisting of agitation, irritability, lethargy, oversleeping, poor feeding, and poor weight gain.

In some embodiments, the daily dose administered to the subject is decreased by 10 mg if the relative infant dose is above the maximum relative infant dose, or if the child shows abnormal behavior.

In some embodiments, the method further comprises administration of a second therapeutic agent.

In some embodiments, Compound (1), or the pharmaceutically acceptable salt of Compound (1), is re-administered to the subject in response to a recurrence of depression symptoms after completion of an initial treatment period. In some embodiments, there is at least a 6 week interval between the last dose of the initial treatment period and the first dose of the re-administration.

In one aspect the disclosure provides a method of treating postpartum depression (PPD) in a human female subject during the subject's postnatal period, the method comprising:
a) administering to the subject about 30 mg to about 50 mg of Compound (1) once a day for about 14 days:

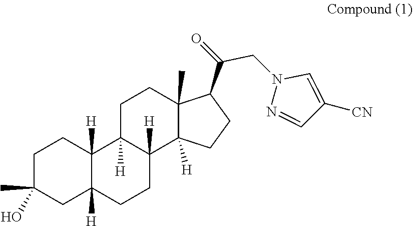

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

In one aspect the disclosure provides a method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days:

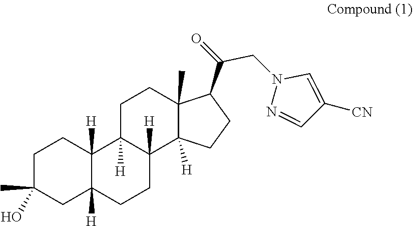

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

In one aspect the disclosure provides a method of treating major depressive disorder (MDD) in a human female subject, the method comprising:
a) administering to the subject about 30 mg to about 50 mg of Compound (1) once a day for about 14 days:

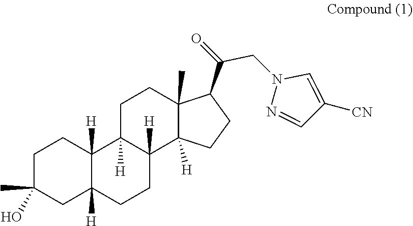

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

In one aspect the disclosure provides a method of treating major depressive disorder (MDD) in a human female, the method comprising:
a) administering to the subject a pharmaceutically acceptable salt of Compound (1) at a dose equivalent to about 30 mg to about 50 mg of the free base compound once a day for about 14 days:

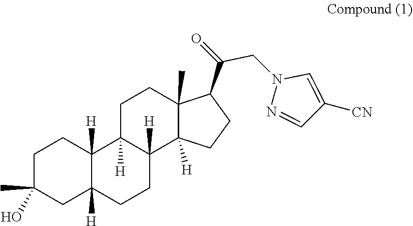

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

In one aspect the disclosure provides a method of treating postpartum depression (PPD) in a human female subject during the subject's postnatal period, the method comprising:
a) administering to the subject about 30 mg to about 50 mg of Compound (1) once a day for about 14 days:

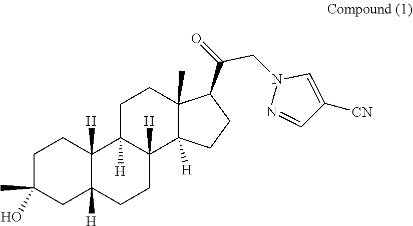

for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

In one aspect the disclosure provides a method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days:

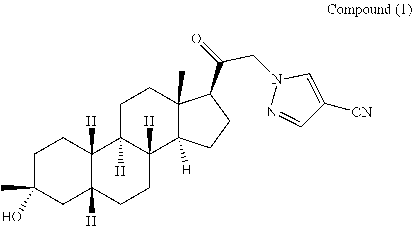

for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

In one aspect the disclosure provides a method of treating major depressive disorder (MDD) in a human female subject, the method comprising:
a) administering to the subject about 30 mg to about 50 mg of Compound (1) once a day for about 14 days:

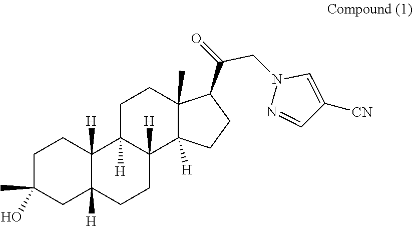

for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

In one aspect the disclosure provides a method of treating major depressive disorder (MDD) in a human female subject, the method comprising:
a) administering to the subject a pharmaceutically acceptable salt of Compound (1) at a dose equivalent to about 30 mg to about 50 mg of the free base compound once a day for about 14 days:

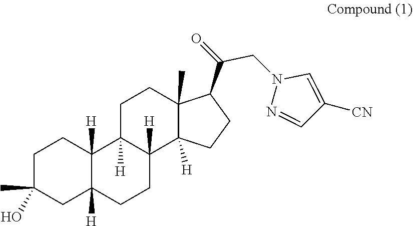

for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

In some embodiments, the PPD is PPD with elevated anxiety. In some embodiments, the MDD is MDD with elevated anxiety.

In some embodiments, Compound (1) is administered at a dose of about 50 mg or the pharmaceutically acceptable salt of Compound (1) is administered at a dose equivalent to about 50 mg of the free base compound. In some embodiments, Compound (1) is administered at a dose of about 40 mg or the pharmaceutically acceptable salt of Compound (1) is administered at a dose equivalent to about 40 mg of the free base compound. In some embodiments, Compound (1) is administered at a dose of about 30 mg or the pharmaceutically acceptable salt of Compound (1) is administered at a dose equivalent to about 30 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered with food.

In some embodiments, Compound (1), or the pharmaceutically acceptable salt of Compound (1), is administered once a day at night.

In some embodiments, the subject is treatment naïve.

In some embodiments, the subject has been on a stable dose of an additional antidepressant for at least 30 days or for at least 60 days prior to the beginning of the treatment period.

In some embodiments, the method further comprises administration of second therapeutic agent.

In some embodiments, Compound (1), or the pharmaceutically acceptable salt of Compound (1), is re-administered to the subject in response to a recurrence of depression symptoms after completion of an initial treatment period. In some embodiments, there is at least a 6 week interval between the last dose of the initial treatment period and the first dose of the re-administration.

In some embodiments, the maximum relative infant dose (RID) is at most about 0.5% of the daily dose administered to the subject. In some embodiments, the maximum relative infant dose (RID) is at most about 0.4% of the daily dose administered to the subject. In some embodiments, the maximum relative infant dose (RID) is at most about 0.357% of the daily dose administered to the subject.

In some embodiments, the abnormal behavior is selected from the group consisting of agitation, irritability, lethargy, oversleeping, poor feeding, and poor weight gain.

### DETAILED DESCRIPTION

### I. Definitions

As used herein, “Compound (1)” refers to the compound having the formula (or structure):

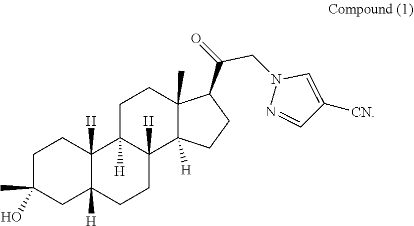

Compound (1) is also known as zuranolone, 3α-hydroxy-3β-methyl-21-(4-cyanopyrazol-1-yl)-5β-19-norpregnan-20-one, and by its IUPAC name: 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile (CAS Registry Number 1632051-40-1). A method of chemically synthesizing Compound (1), was described in U.S. Pat. No. 9,512,165 and PCT Application Publication No. WO 2014/169833; the entire contents of the aforementioned applications are incorporated herein by reference in their entireties. Several crystalline forms of Compound (1) and methods of preparing said forms were described in U.S. Pat. No. 11,236,121; U.S. Patent Application Publication No. US 2019/0177359; and PCT Application Publication No. WO 2018/039378; the entire contents of the aforementioned applications are incorporated herein by reference in their entireties. Pharmaceutical compositions of Compound (1) and methods of preparing said compositions were described in PCT Application Publication No. WO 2022/020363A9 and in U.S. application Ser. No. 17/579,541; the entire contents of the aforementioned applications are each incorporated herein by reference in its entirety.

Compound (1) is a neuroactive steroid that has been shown to be a positive allosteric modulator of GABAA receptors that target synaptic and extrasynaptic GABAA receptors. As a positive allosteric modulator of GABAA receptors, Compound (1) serves as a therapeutic agent to treat CNS related disorders, e.g., depression, postpartum depression and major depressive disorder and to treat neurological conditions, e.g., essential tremor, epilepsy, and Parkinson's disease.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms refer to crystalline modifications comprising a given substance (e.g., Compound (1)), including single-component crystal forms and multiple-component crystal forms, and including, but not limited to, polymorphs, solvates, hydrates, and salts.

The term “substantially crystalline” refers to forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In some embodiments, the particular weight percent of crystallinity is at least 90%. In some embodiments, the particular weight percent of crystallinity is at least 95%. In some embodiments, Compound (1) can be a substantially crystalline sample of any of the crystalline forms described herein (e.g., crystalline Forms A and C) and/or PCT Application Publication No. WO 2018/039378; the entire contents of the aforementioned application are incorporated herein by reference in its entirety.

The term “substantially pure” relates to the composition of a specific crystalline form (e.g., a crystalline form of Compound (1)) that may be at least a particular weight percent free of impurities and/or other solid forms. Particular weight percentages may include 70%, 75%, 80%, 85%, 90%, 95%, 99%, or any percentage between 70% and 100%. In some embodiments, Compound (1) can be a substantially pure sample of any of the crystalline forms described herein, (e.g., crystalline Forms A and C). In some embodiments, Compound (1) can be substantially pure Form A. In some embodiments, Compound (1) can be substantially pure Form C.

As used herein, “XRPD” refers to X-ray powder diffraction. An XRPD pattern is an x-y graph with 2Q (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

The term “characteristic peaks” when referring to the peaks in an XRPD pattern of a crystalline form of a given chemical entity (e.g., a crystalline form of Compound (1)) refers to a collection of specific diffraction peaks whose values span a range of 20 values (e.g., 0° to 40°) that are, as a whole, unique to that specific crystalline form.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

The chemical elements are identified in accordance with the Periodic Table of the Elements, CAS version, Handbook of Chemistry and Physics, 75ili Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5ili Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

As used herein, a “child” is a human below the age of 18 years old. In some embodiments, a child is a human being aged 0 to 18 years old, or 0 to 5 years old, or 0 to 4 years old, or 0 to 3 years old, or 0 to 2 years, or 0 to 18 months, or 0 to 12 months, or 0 to 6 months. In some embodiments, a child is a human being aged 0 to 18 months. In some embodiments, a child is a human being aged 0 to 12 months. In some embodiments, a child is a human being aged 0 to 6 months.

The terms “disease”, “disorder”, and “condition” are used interchangeably herein.

As used herein, the term “dose equivalent” means a bioequivalent dose. For example, the dose equivalent of a pharmaceutically acceptable salt of Compound (1) for a 50 mg dose of Compound (1) is the amount of the pharmaceutically acceptable salt (by weight) needed to provide a bioequivalent dose to the 50 mg dose of the free base of Compound (1).

As used herein, an “effective amount” of a compound (or pharmaceutically acceptable salt thereof) refers to an amount sufficient to elicit the desired biological response, e.g., to treat a CNS-related disorder, e.g., depression, e.g., postpartum depression (PPD), major depressive disorder (MDD), postpartum depression (PPD) with elevated anxiety, or major depressive disorder (MDD) with elevated anxiety. As will be appreciated by those of ordinary skill in this art, the effective amount of a compound (or pharmaceutically acceptable salt thereof) of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, an “episodic dosing regimen” is a dosing regimen wherein a compound or a composition comprising a compound is administered to a subject for a finite period of time in response to the diagnosis of a disorder or symptom thereof, e.g., a diagnosis or symptom of depression or an episode of major depressive disorder. In some embodiments, the major depressive disorder is moderate major depressive disorder. In some embodiments, the major depressive disorder is severe major depressive disorder. In some embodiments, the compound is formulated as individual dosage units, each unit comprising Compound (1) and one or more suitable pharmaceutical excipient. In some embodiments, the episodic dosing regimen has a duration of a plurality of weeks, e.g., about 8 weeks. In contrast with chronic administration as defined herein, episodic dosing of a compound occurs over a finite period of time, e.g., from about 2 weeks to about 8 weeks, in response to a diagnosis or recurrence of a disorder, e.g., depression, or a symptom thereof. In some embodiments, episodic dosing occurs once per day across a plurality of weeks, e.g., from about 2 weeks to about 6 weeks. In one embodiment, the episodic dosing has a duration of two weeks. In some embodiments, more than one episodic dosing regimen, but no more than 3 episodic dosing regimens, is administered to the subject, e.g., two or more episodic regimens over a period of 12 months.

As used herein, the term “modulation” refers to the inhibition or potentiation of GABAA receptor function. A “modulator” (e.g., a compound or pharmaceutically acceptable salt thereof that modulates GABAA receptor function) may be, for example, an agonist, partial agonist, antagonist, or partial antagonist of the GABAA receptor.

“MDD with elevated anxiety” or “MDD with anxious distress” are used interchangeably and refer to subjects with MDD who present elevated anxiety as a symptom of their depression. In some embodiments, MDD with elevated anxiety is characterized by a HAM-D Anxiety/Somatization Subscale score of at least 7 at baseline (e.g., prior to administration of Compound (1) or a pharmaceutically acceptable salt thereof). In some embodiments, MDD with elevated anxiety is characterized by a HAM-A total score of at least 17 at baseline (e.g., prior to administration of Compound (1) or a pharmaceutically acceptable salt thereof). In some embodiments, MDD with elevated anxiety is characterized by a HAM-A total score of at least 18 at baseline. In some embodiments, MDD with elevated anxiety is characterized by a HAM-A total score of at least 20 at baseline. “PPD with elevated anxiety” or “PPD with anxious distress” are used interchangeably and refer to subjects with PPD who present elevated anxiety as a symptom of their depression. In some embodiments, PPD with elevated anxiety is characterized by a HAM-D Anxiety/Somatization Subscale score of at least 7 at baseline (e.g., prior to administration of Compound (1) or a pharmaceutically acceptable salt thereof). In some embodiments, PPD with elevated anxiety is characterized by a HAM-A total score of at least 17 at baseline (e.g., prior to administration of Compound (1) or a pharmaceutically acceptable salt thereof). In some embodiments, PPD with elevated anxiety is characterized by a HAM-A total score of at least 18 at baseline. In some embodiments, PPD with elevated anxiety is characterized by a HAM-A total score of at least 20 at baseline.

In other embodiments, “elevated anxiety” is characterized by a HAM-A score based on the HAM-A anxiety items and somatic items. In some embodiments, “elevated anxiety” is characterized by a HAM-A score based on the HAM-A anxiety items. In some embodiments, “elevated anxiety” is characterized by a HAM-D score based on the following HAM-D items: psychic anxiety, somatic anxiety, GI somatic symptoms, and/or general somatic symptoms. In some embodiments, “elevated anxiety” is characterized by a HAM-D score based on the following HAM-D item: psychic anxiety. In some embodiments, “elevated anxiety” is characterized by a HAM-D score based predominately on the items evaluating somatic symptoms of depression. In some embodiments, “elevated anxiety” is characterized by a HAM-D score based predominately on the items evaluating anxiety symptoms of depression. In some embodiments, “elevated anxiety” is characterized by a HAM-D Anxiety/Somatization Subscale score based predominately on the items evaluating somatic symptoms of depression. In some embodiments, “elevated anxiety” is characterized by a HAM-D Anxiety/Somatization Subscale score based predominately on the items evaluating anxiety symptoms of depression. In some embodiments, “elevated anxiety” is characterized by a MADRS score based predominately on the items evaluating the somatic symptoms of depression. In some embodiments, “elevated anxiety” is characterized by a MADRS score based predominately on the items evaluating the anxiety symptoms of depression.

As used herein, and unless otherwise specified, a “therapeutically effective amount” of a compound (or pharmaceutically acceptable salt thereof) is an amount sufficient to provide a therapeutic benefit in the treatment of a disease, disorder or condition, or to delay or minimize one or more symptoms associated with the disease, disorder or condition. A therapeutically effective amount of a compound (or pharmaceutically acceptable salt thereof) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present disclosure contemplates administration of Compound (1) or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, “solid dosage form” means a pharmaceutical dose(s) in solid form, e.g., tablets, capsules, granules, powders, sachets, reconstitutable powders, dry powder inhalers and chewables.

A “subject” can also be a human female (e.g., a female of any age group) who is pregnant, about to give birth, or has given birth. The terms “human,” “patient,” and “subject” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

As used herein, “treatment naïve” refers to a subject that has not been previously treated with the additional antidepressant within the current depressive episode. “Treatment naïve” also refers to a subject that has not taken any antidepressant within at least 30 days prior or within at least 60 days prior to the start of treatment (e.g., Day 1). In some embodiments, the treatment naïve subject has not taken any antidepressant within at least 30 days prior to the start of treatment. In some embodiments, the treatment naïve subject has not taken any antidepressant within at least 60 days prior to the start of treatment.

As used herein, the term “unit dosage form” is defined to refer to the form in which Compound (1) is administered to the subject. In some embodiments, the unit dosage form can be, for example, a pill, capsule, or tablet. In some embodiments, the unit dosage form is a capsule. In some embodiments, the typical amount of Compound (1) in a unit dosage form useful in the disclosure is about 10 mg to about 100 mg, about 20 mg to about 55 mg, or about 30 mg to about 50 mg (e.g., about, 10 mg, about 15 mg, about 20 mg, about 25 mg, about 30 mg, about 35 mg, about 40 mg, about 45 mg, about 50 mg, or about 55 mg).

In some embodiments, the unit dosage form comprises about 30 mg of Compound (1) and is in the form of a capsule. In some embodiments, the unit dosage form comprises about 50 mg of Compound (1) and is in the form of a capsule. In some embodiments, the unit dosage form comprises about 40 mg of Compound (1) and is in the form of a capsule. In some embodiments, the unit dosage form comprises about 45 mg of Compound (1) and is in the form of a capsule. In some embodiments, the unit dosage form comprises about 20 mg of Compound (1) and is in the form of a capsule. In some embodiments, the unit dosage form comprises about 10 mg of Compound (1) and is in the form of a capsule. In some embodiments, the unit dosage form comprises about 15 mg of Compound (1) and is in the form of a capsule. In some embodiments, the unit dosage form comprises about 25 mg of Compound (1) and is in the form of a capsule. In some embodiments, one or more capsules, which comprise about 30 mg or 45 mg of Compound (1), are administered to a subject once per day. In some embodiments, three capsules together comprise the 30 mg of Compound (1). In some embodiments, three capsules together comprises the 45 mg of Compound (1).

In some embodiments, administering Compound (1) improves cognitive function. In some embodiments, the cognitive function refers to a collection of mental tasks and functions, including but not limited to: memory (e.g., semantic, episodic, procedural, priming, or working); orientation; language; problem solving; visual perception, construction, and integration; planning; organizational skills; selective attention; inhibitory control; and ability to mentally manipulate information. In one embodiment, the cognitive function is one or more selected from the group consisting of memory (e.g., semantic, episodic, procedural, priming, or working); orientation; language; problem solving; visual perception, construction, and integration; planning; organizational skills; selective attention; inhibitory control; and ability to mentally manipulate information. Measures of cognitive functioning include assessment tools designed to measure, for example: (a) general intelligence, (b) nonverbal intelligence, (c) achievement, (d) attention/executive functioning, (e) memory and learning, (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered).

### II. Methods of Treatment

### Postpartum Depression (PPD)

In one aspect, the present disclosure is directed to methods of treating postpartum depression (PPD) in a human female subject during the subject's postnatal period. In some embodiments, the PPD is PPD with elevated anxiety.

Postpartum depression (PPD), also called postnatal depression, is a type of mood disorder associated with childbirth. Postpartum depression (PPD) is generally known in the art.

Postnatal depression (PND) also referred to as postpartum depression (PPD), refers to a type of clinical depression that affects women after childbirth. Symptoms can include sadness, fatigue, changes in sleeping and eating habits, reduced sexual desire, crying episodes, anxiety, and irritability. In one embodiment, the PND is a treatment-resistant depression. In one embodiment, the PND is refractory depression.

In one embodiment, a subject having PND also experienced depression, or a symptom of depression during pregnancy. This depression can be referred to as perinatal depression. In an embodiment, a subject experiencing perinatal depression is at increased risk of experiencing PND.

PPD is identified as the most common psychiatric illness to occur in the puerperium (O'Hara M W, Wisner K L. Best Pract Res Clin Obstet Gynaecol. 2014; 28(1):3-12); and it can occur during the third trimester or after giving birth. If untreated, PPD can have devastating consequences for the woman and her family. In some embodiments, PPD is characterized by significant functional impairment for the mother due to sadness and depressed mood, loss of interest in daily activities, changes in eating and sleeping habits, fatigue and decreased energy, inability to concentrate, and feelings of worthlessness, shame, or guilt. Postpartum depression also carries an increased risk for suicide, which is the leading cause of maternal death following childbirth in developed countries.

Professional health organizations differ in their definition of PPD onset. For example, the American Psychiatric Association characterizes PPD as having an onset during pregnancy or within 4 weeks of delivery (DSM-5). The American College of Obstetricians and Gynecologists characterizes PPD as having an onset during pregnancy or within 12 months postpartum (ACOG Updated December 2021). The World Health Organization characterizes PPD as having an onset within 12 months postpartum (International Classification of Diseases 10^{th} edition (ICD-10)). Accordingly, in some embodiments, the diagnosis of the PPD treated by the methods described herein can be characterized as defined by the DSM-5. In some embodiments, the diagnosis of the PPD treated by the methods described herein can be characterized as defined by the ACOG. In some embodiments, the diagnosis of the PPD treated by the methods described herein can be characterized as defined by the ICD-10.

In some embodiments, the diagnosis of the PPD treated by the methods described herein can be characterized as defined by the Diagnostic and Statistical Manual of Mental Disorders, 5^{th} Edition (DSM-5), that is as a MDD with peripartum onset, as described below.

### Depressive Disorders

Depressive disorders include disruptive mood dysregulation disorder, major depressive disorder (including major depressive episode), persistent depressive disorder (dysthymia), premenstrual dysphoric disorder, substance/medication-induced depressive disorder, depressive disorder due to another medical condition, other specified depressive disorder, and unspecified depressive disorder. The common feature of all of these disorders is the presence of sad, empty, or irritable mood, accompanied by somatic and cognitive changes that significantly affect the individual's capacity to function. What differs among them are issues of duration, timing, or presumed etiology.

Major depressive disorder represents the classic condition in this group of disorders. It is characterized by discrete episodes of at least 2 weeks' duration (although most episodes last considerably longer) involving clear-cut changes in affect, cognition, and neurovegetative functions and inter-episode remissions. A discrete episode of major depressive disorder may be referred to as a “major depressive episode” or “depressive episode”.

### Major Depressive Disorder (MDD)

In some embodiments, MDD is also known as depression or clinical depression and it is a mood disorder that causes a persistent feeling of sadness and loss of interest.

In some embodiments, MDD is defined and diagnosed according to the DSM-5, for example, MDD is diagnosed according to Criterion A, as described below.

Criterion A. Five (or more) of the following symptoms have been present during the same 2-week period and represent a change from previous functioning; at least one of the symptoms is either (1) depressed mood or (2) loss of interest or pleasure.
1. Depressed mood most of the day, nearly every day, as indicated by either subjective report (e.g., feels sad, empty, hopeless) or observation made by others (e.g., appears tearful). (Note: In children and adolescents, can be irritable mood.)
2. Markedly diminished interest or pleasure in all, or almost all, activities most of the day, nearly every day (as indicated by either subjective account or observation).
3. Significant weight loss when not dieting or weight gain (e.g., a change of more than 5% of body weight in a month), or decrease or increase in appetite nearly every day (Note: In children, consider failure to make expected weight gain.)
4. Insomnia or hypersomnia nearly every day.
5. Psychomotor agitation or retardation nearly every day (observable by others, not merely subjective feelings of restlessness or being slowed down).
6. Fatigue or loss of energy nearly every day.
7. Feelings of worthlessness or excessive or inappropriate guilt (which may be delusional) nearly every day (not merely self-reproach or guilt about being sick).
8. Diminished ability to think or concentrate, or indecisiveness, nearly every day (either by subjective account or as observed by others).
9. Recurrent thoughts of death (not just fear of dying), recurrent suicidal ideation without a specific plan, or a suicide attempt or a specific plan for committing suicide.

Criteria B-E, described below, are additional descriptions of MDD and may be considered for describing or diagnosing MDD, but are not required.

Criterion B. The symptoms cause clinically significant distress or impairment in social, occupational, or other important areas of functioning.

Criterion C. The episode is not attributable to the physiological effects of a substance or to another medical condition.

Criteria A-C can represent a major depressive episode.

Criterion D. The occurrence of the major depressive episode is not better explained by schizoaffective disorder, schizophrenia, schizophreniform disorder, delusional disorder, or other specified and unspecified schizophrenia spectrum and other psychotic disorders.

Criterion E. There has never been a manic episode or a hypomanic episode.

In some embodiments, a major depressive episode (MDE) is a period characterized by the symptoms described above.

In some embodiments, MDD is a clinical course that is characterized by one or more major depressive episodes (MDE) in a subject.

In some embodiments, MDD is diagnosed according to Criteria A-C, as described above. In some embodiments, MDD is diagnosed according to Criteria A-E, as described above.

### Diagnostic Features

The criterion symptoms for major depressive disorder must be present nearly every day to be considered present, with the exception of weight change and suicidal ideation. Depressed mood must be present for most of the day, in addition to being present nearly every day. Often insomnia or fatigue is the presenting complaint, and failure to probe for accompanying depressive symptoms will result in under diagnosis. Sadness may be denied at first but may be elicited through interview or inferred from facial expression and demeanor. With individuals who focus on a somatic complaint, clinicians should determine whether the distress from that complaint is associated with specific depressive symptoms. Fatigue and sleep disturbance are present in a high proportion of cases; psychomotor disturbances are much less common but are indicative of greater overall severity, as is the presence of delusional or near-delusional guilt.

The essential feature of a major depressive episode is a period of at least 2 weeks during which there is either depressed mood or the loss of interest or pleasure in nearly all activities (Criterion A above). In children and adolescents, the mood may be irritable rather than sad. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation or suicide plans or attempts. To count toward a major depressive episode, a symptom must either be newly present or must have clearly worsened compared with the person's pre-episode status. The symptoms must persist for most of the day, nearly every day, for at least 2 consecutive weeks. The episode must be accompanied by clinically significant distress or impairment in social, occupational, or other important areas of functioning. For some individuals with mild episodes, functioning may appear to be normal but requires markedly increased effort.

Sleep disturbance may take the form of either difficulty sleeping or sleeping excessively (Criterion A4). When insomnia is present, it typically takes the form of middle insomnia (e.g., waking up during the night and then having difficulty returning to sleep) or terminal insomnia (e.g., waking too early and being unable to return to sleep). Initial insomnia (e.g., difficulty falling asleep) may also occur. Individuals who present with over-sleeping (hypersomnia) may experience prolonged sleep episodes at night or increased daytime sleep. Sometimes the reason that the individual seeks treatment is for the disturbed sleep.

### With Peripartum Onset (Specifier for Depressive Disorders Per the DSM-5)

This specifier can be applied to the current or, if full criteria are not currently met for a major depressive episode, most recent episode of major depression if onset of mood symptoms occurs during pregnancy or in the 4 weeks following delivery.

Mood episodes can have their onset either during pregnancy or postpartum. Although the estimates differ according to the period of follow-up after delivery, between 3% and 6% of women will experience the onset of a major depressive episode during pregnancy or in the weeks or months following delivery. Fifty percent of “postpartum” major depressive episodes actually begin prior to delivery. Thus, these episodes are referred to collectively as peripartum episodes. Women with peripartum major depressive episodes often have severe anxiety and even panic attacks. Prospective studies have demonstrated that mood and anxiety symptoms during pregnancy, as well as the “baby blues,” increase the risk for a post partum major depressive episode. Peripartum-onset mood episodes can present either with or without psychotic features. Infanticide is most often associated with postpartum psychotic episodes that are characterized by command hallucinations to kill the infant or delusions that the infant is possessed, but psychotic symptoms can also occur in severe post partum mood episodes without such specific delusions or hallucinations.

In some embodiments, the diagnosis of the PPD with elevated anxiety treated by the methods described herein can be characterized as defined by the Diagnostic and Statistical Manual of Mental Disorders, 5^{th} Edition (DSM-5), that is as a MDD with peripartum onset and with anxious distress specifiers, as described below.

### With Elevated Anxiety/Anxious Distress (Specifier for Depressive Disorders Per the DSM-5)

The DSM-5 defines the “anxious distress” specifier as the presence of at least two of the following symptoms during the majority of days of a major depressive episode (MDD) or persistent depressive disorder (dysthymia):
1. Feeling keyed up or tense.
2. Feeling unusually restless.
3. Difficulty concentrating because of worry.
4. Fear that something awful may happen.
5. Feeling that the individual might lose control of himself or herself.

Severity is defined as:
Mild: Two symptoms.
Moderate: Three symptoms.
Moderate-severe: Four or five symptoms.
Severe: Four or five symptoms and with motor agitation.

Anxious distress has been noted as a prominent feature of both bipolar and major depressive disorder in both primary care and specialty mental health settings. High levels of anxiety have been associated with higher suicide risk, longer duration of illness, and greater likelihood of treatment nonresponse.

In some embodiments, the PPD is MDD, with peripartum onset. In some embodiments, the PPD with elevated anxiety is MDD, with peripartum onset, with anxious distress.

Accordingly, one aspect of the present disclosure is directed to a method of treating postpartum depression (PPD) in a human female subject during the subject's postnatal period, the method comprising administering to the subject a therapeutically effective amount of Compound (1):

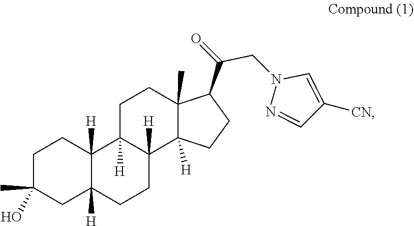

for a treatment period, wherein the subject breastfeeds a child during the treatment period.

Another aspect of the disclosure is directed to a method of treating postpartum depression (PPD) in a human female subject during the subject's postnatal period, the method comprising administering to the subject a therapeutically effective amount a pharmaceutically acceptable salt of Compound (1):

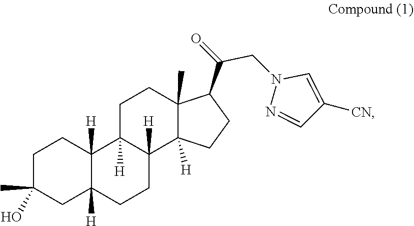

for a treatment period, wherein the subject breastfeeds a child during the treatment period.

Another aspect of the disclosure is directed to a method of treating postpartum depression (PPD) with elevated anxiety in a human female subject during the subject's postnatal period, the method comprising administering to the subject a therapeutically effective amount of Compound (1):

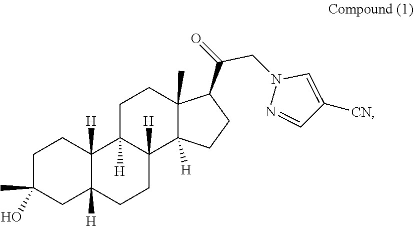

for a treatment period, wherein the subject breastfeeds a child during the treatment period.

Another aspect of the disclosure is directed to a method of treating postpartum depression (PPD) with elevated anxiety in a human female subject during the subject's postnatal period, the method comprising administering to the subject a therapeutically effective amount a pharmaceutically acceptable salt of Compound (1):

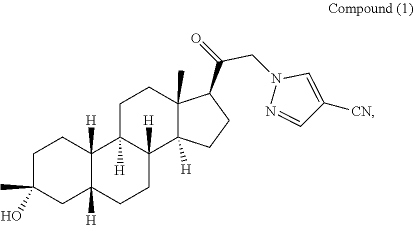

for a treatment period, wherein the subject breastfeeds a child during the treatment period.

In some embodiments, the subject breastfeeds the infant at least 1 time per day. In another embodiment, the subject breastfeeds the infant at least 2 times per day. In another embodiment, the subject breastfeeds the infant at least 3 times per day. In another embodiment, the subject breastfeeds the infant at least 4, 5, 6, 7, 8, 9, 10, 11, or 12 times per day.

In some embodiments, the treatment period is about 2 weeks or about 14 days. In some embodiments, the treatment period is about 2 weeks. In some embodiments, the treatment period is about 14 days.

In some embodiments, Compound (1), or the pharmaceutically acceptable salt of Compound (1), is administered once a day for about 14 days or about 2 weeks. In some embodiments, Compound (1), or the pharmaceutically acceptable salt of Compound (1), is administered once a day for about 14 days. In some embodiments, Compound (1), or the pharmaceutically acceptable salt of Compound (1), is administered once a day for about 2 weeks.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg to about 55 mg. In some embodiments, Compound (1) is administered at a dose of about 30 mg to about 50 mg. In some embodiments, Compound (1) is administered at a dose of about 45 mg to about 55 mg. In some embodiments, Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg. In some embodiments, Compound (1) is administered at a dose of about 30 mg.

In some embodiments, Compound (1) is administered at a dose of about 10 mg to about 100 mg once a day. In some embodiments, Compound (1) is administered at a dose of about 15 mg to about 75 mg once a day. In some embodiments, Compound (1) is administered at a dose of about 20 mg to about 60 mg once a day. In some embodiments, Compound (1) is administered at a dose of about 20 mg to about 55 mg once a day. In some embodiments, Compound (1) is administered at a dose of about 30 mg to about 50 mg once a day. In some embodiments, Compound (1) is administered at a dose of about 45 mg to about 55 mg once a day. In some embodiments, Compound (1) is administered at a dose of about 20 mg, about 25 mg, about 30 mg, about 35 mg, about 40 mg, about 45 mg, about 50 mg, about 55 mg, or about 60 mg once a day. In some embodiments, Compound (1) is administered at a dose of about 50 mg once a day. In some embodiments, Compound (1) is administered at a dose of about 40 mg once a day. In some embodiments, Compound (1) is administered at a dose of about 30 mg once a day.

In some embodiments, Compound (1) is administered at a dose of about 20 mg to about 55 mg once a day for about 2 weeks or about 14 days. In some embodiments, Compound (1) is administered at a dose of about 30 mg to about 50 mg once a day for about 2 weeks or about 14 days. In some embodiments, Compound (1) is administered at a dose of about 45 mg to about 55 mg once a day for about 2 weeks or about 14 days. In some embodiments, Compound (1) is administered at a dose of about 50 mg once a day for less than 2 weeks. In some embodiments, Compound (1) is administered at a dose of about 50 mg once a day for about 2 weeks. In some embodiments, Compound (1) is administered at a dose of about 50 mg once a day for about 14 days. In some embodiments, Compound (1) is administered at a dose of about 40 mg once a day for less than 2 weeks. In some embodiments, Compound (1) is administered at a dose of about 40 mg once a day for about 2 weeks. In some embodiments, Compound (1) is administered at a dose of about 40 mg once a day for about 14 days. In some embodiments, Compound (1) is administered at a dose of about 30 mg once a day for less than 2 weeks. In some embodiments, Compound (1) is administered at a dose of about 30 mg once a day for about 2 weeks. In some embodiments, Compound (1) is administered at a dose of about 30 mg once a day for about 14 days.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 45 mg to about 55 mg of the free base compound. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound. In some embodiments, the pharmaceutically acceptable salt of Compound (1) is administered at a dose equivalent of about 30 mg of the free base compound.

In some embodiments, the pharmaceutically acceptable salt of Compound (1) is administered at a dose equivalent of about 10 mg to about 100 mg of the free base compound once a day. In some embodiments, the pharmaceutically acceptable salt of Compound (1) is administered at a dose equivalent of about 15 mg to about 75 mg of the free base compound once a day. In some embodiments, the pharmaceutically acceptable salt of Compound (1) is administered at a dose equivalent of about 20 mg to about 60 mg of the free base compound once a day. In some embodiments, the pharmaceutically acceptable salt of Compound (1) is administered at a dose equivalent of about 20 mg to about 55 mg of the free base compound once a day. In some embodiments, the pharmaceutically acceptable salt of Compound (1) is administered at a dose equivalent of about 30 mg to about 50 mg of the free base compound once a day. In some embodiments, the pharmaceutically acceptable salt of Compound (1) is administered at a dose equivalent of about 45 mg to about 55 mg of the free base compound once a day. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) is administered at a dose equivalent of about 50 mg of the free base compound once a day. In some embodiments, the pharmaceutically acceptable salt of Compound (1) is administered at a dose equivalent of about 40 mg of the free base compound once a day. In some embodiments, the pharmaceutically acceptable salt of Compound (1) is administered at a dose equivalent of about 30 mg of the free base compound once a day.

In some embodiments, the pharmaceutically acceptable salt of Compound (1) is administered at a dose equivalent of about 20 mg to about 55 mg of the free base compound once a day for about 2 weeks or about 14 days. In some embodiments, the pharmaceutically acceptable salt of Compound (1) is administered at a dose equivalent of about 30 mg to about 50 mg of the free base compound once a day for about 2 weeks or about 14 days. In some embodiments, the pharmaceutically acceptable salt of Compound (1) is administered at a dose equivalent of about 45 mg to about 55 mg of the free base compound once a day for about 2 weeks or about 14 days. In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 2 weeks. In some embodiments, the pharmaceutically acceptable salt of Compound (1) is administered at a dose equivalent of about 50 mg of the free base compound once a day for about 14 days. In some embodiments, the pharmaceutically acceptable salt of Compound (1) is administered at a dose equivalent of about 40 mg of the free base compound once a day for less than 2 weeks. In some embodiments, the pharmaceutically acceptable salt of Compound (1) is administered at a dose equivalent of about 40 mg of the free base compound once a day for about 2 weeks. In some embodiments, the pharmaceutically acceptable salt of Compound (1) is administered at a dose equivalent of about 40 mg of the free base compound once a day for about 14 days. In some embodiments, the pharmaceutically acceptable salt of Compound (1) is administered at a dose equivalent of about 30 mg of the free base compound once a day for less than 2 weeks. In some embodiments, the pharmaceutically acceptable salt of Compound (1) is administered at a dose equivalent of about 30 mg of the free base compound once a day for about 2 weeks. In some embodiments, the pharmaceutically acceptable salt of Compound (1) is administered at a dose equivalent of about 30 mg of the free base compound once a day for about 14 days.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered chronically.

In some embodiments, Compound (1), or the pharmaceutically acceptable salt of Compound (1), is administered in one or more capsules. In some embodiments, the therapeutically effective amount is administered across two capsules. In some embodiments, the therapeutically effective amount is administered across three capsules.

In some embodiments, Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps. In some embodiments, the subject is administered Compound (1), or the pharmaceutically acceptable salt of Compound (1), once a day at night. In some embodiments, the subject is administered Compound (1), or the pharmaceutically acceptable salt of Compound (1), once a day no later than 1 hour before the patient sleeps. In some embodiments, the subject is administered Compound (1), or the pharmaceutically acceptable salt of Compound (1), once a day no later than 15 minutes before the patient sleeps.

In some embodiments, Compound (1) is in a crystalline form. In some embodiments, the crystalline form of Compound (1) is any crystalline form disclosed in PCT Application Publication No. WO 2018/039378; the entire contents of the aforementioned application are incorporated herein by reference in its entirety.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 20. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 20. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the crystalline form of Compound (1) comprises a mixture of two or more crystalline forms.

In some embodiments, the subject is treatment naïve. In some embodiments, the subject has not received any antidepressant treatment within at least 30 days prior to the administration of Compound (1), or the pharmaceutically acceptable salt of Compound (1). In some embodiments, the subject has not received any antidepressant treatment within at least 60 days prior to the administration of Compound (1), or the pharmaceutically acceptable salt of Compound (1).

In some embodiments, the subject has been on a stable dose of an additional antidepressant for at least 30 days or for at least 60 days prior to the beginning of the treatment period. In some embodiments, the subject has been on a stable dose of an additional antidepressant for at least 30 days prior to the beginning of the treatment period. In some embodiments, the subject has been on a stable dose of an additional antidepressant for at least 60 days prior to the beginning of the treatment period.

In some embodiments, the breast milk of the subject is monitored to determine relative infant dose (RID) of Compound (1), or the pharmaceutically acceptable salt of Compound (1), in the breast milk, and the daily dose of Compound (1) is adjusted to produce less than a maximum relative infant dose (RID). RID estimates infant drug exposure via breast milk. The RID uses a known milk concentration and compares it to either an infant therapeutic dose or the weight-adjusted maternal dose when an infant dose is not well established. Typically, breastfeeding is considered acceptable when the relative infant dose is <10%. Additional considerations include the gestational and postnatal age of the infant, the actual amount of milk being ingested (less in the first couple days of life and when weaning), properties of the specific maternal medication, medical conditions of the infant, and medications the infant is receiving therapeutically.

In some embodiments, the maximum relative infant dose (RID) is at most about 0.5% of the daily dose administered to the subject. In some embodiments, the maximum relative infant dose (RID) is at most about 0.4% of the daily dose administered to the subject. In some embodiments, the maximum relative infant dose (RID) is at most about 0.357% of the daily dose administered to the subject.

In some embodiments, the child is monitored for abnormal behavior. In some embodiments, the abnormal behavior is selected from the group consisting of agitation, irritability, lethargy, oversleeping, poor feeding, and poor weight gain.

In some embodiments, the daily dose administered to the subject is decreased by 10 mg if the relative infant dose is above the maximum relative infant dose (RID), or if the child shows abnormal behavior. In some embodiments, the daily dose administered to the subject is decreased by 10 mg if the relative infant dose is above the maximum relative infant dose (RID). In some embodiments, the daily dose administered to the subject is decreased by 10 mg if the child shows abnormal behavior.

In some embodiments, Compound (1), or the pharmaceutically acceptable salt of Compound (1), is re-administered to the subject in response to a recurrence of depression symptoms after completion of initial treatment period. In some embodiments, there is at least a 6 week interval between the last dose of the initial treatment period and the first dose of the re-administration. In some embodiments, each of the initial treatment period and re-administration occurs for about 14 days or about 2 weeks.

In some embodiments, the method further comprises administration of a second therapeutic agent.

In some embodiments, the subject is identified to have postpartum depression through a screening method (e.g., Edinburgh Postnatal Depression Scale (EPDS), e.g., a score of 10 or more on the EPDS, a score of 13 or more on the EPDS). In some embodiments, the subject is identified to have postpartum depression through screening instruments such as Patient Health Questionnaire (PHQ) in various forms or the Hospital Anxiety and Depression Scales or Geriatric Depression Scale.

In some embodiments, the subject is disabled or has poor health status due to medical illness, complicated grief, chronic sleep disturbance, loneliness, or history of depression. In some embodiments, the subject has poor self-esteem, child-care stress, prenatal anxiety, life stress, decreased social support, single/unpartnered relationship status, history of depression, difficult infant temperament, previous postpartum depression, lower socioeconomic status, or unintended pregnancy. In some embodiments, the subject has hyperemesis gravidarum (e.g., severe form of morning sickness, e.g., preventing adequate intake of food and fluids). In some embodiments, the subject has had a complication in pregnancy (e.g., emergency C-sections, pre-eclampsia, hospitalization during pregnancy, concern about fetal distress and admission of the child to special care (NICU), the child was in the NICU). In some embodiments, the subject has had emotionally painful or stressful experiences around pregnancy, childbirth, or early parenting (e.g., the subject was treated for infertility, had a previous miscarriage or other pregnancy loss, delivery of multiples, special needs, colic or difficult temperament child, had difficulty feeding). In some embodiments, the subject has had a history of domestic violence, sexual or other abuse (e.g., abused as a child or as an adult). In some embodiments, the subject has had a traumatic childhood (e.g., loss of a parent, troubling relationship with parent). In some embodiments, the subject has stress (e.g., loss of someone close, job loss, financial hardship, divorce, strain in a relationship, house move). In some embodiments, the subject has lack of social support. In some embodiments, the subject has a perfectionist or controlling personality.

In some embodiments, the subject has given birth. In some embodiments, the subject has given birth at least 12 weeks prior to start of treatment. In some embodiments, the subject is due to give birth. In some embodiments, the subject is due to give birth in 9, 8, 7, 6, 5, 4, 3, 2, or 1 months; 4, 3, 2, or 1 weeks; or 7, 6, 5, 4, 3, 2, or 1 days. In some embodiments, the subject is in her third trimester of pregnancy. In some embodiments, the subject has an attribute, characteristic, or exposure (that increases the likelihood of developing a disorder as described herein, e.g., neuroactive steroid deficiency). In some embodiments, the subject has reached term pregnancy (e.g., early term (e.g., between 37 weeks and 38 weeks and 6 days); full term (e.g., between 39 weeks and 40 weeks and 6 days); late term (e.g., between 41 weeks and 41 weeks and 6 days); or post-term (e.g., 42 weeks and beyond)) or has given early term, full term, late term, or post-term birth.

In some embodiments, the method provides therapeutic effect (e.g., as measured by reduction in Hamilton Depression Score (HAM-D)) within about 45, about 21, about 15, about 8, or about 3 days. In some embodiments, the therapeutic effect is a decrease from baseline in HAM-D score at the end of a treatment period (e.g., about 45, about 21, about 15, about 8, or about 3 days after beginning administration or episodic dosing). In some embodiments, the decrease from baseline in HAM-D score is from severe (e.g., HAM-D score of 24 or greater; or a score of 26 or greater) to symptom-free, e.g., remission of depression (e.g., HAM-D score of 7 or lower). In some embodiments, the decrease from baseline in HAM-D score is from severe (e.g., HAM-D score of 24 or greater; or a score of 26 or greater) to normal or mild depression (e.g., HAM-D score of 7 or lower; or HAM-D score of 18-13).

In some embodiments, the method provides therapeutic effect (e.g., as measured by reduction in Montgomery-Asberg Depression Rating Scale (MADRS)) within about 45, about 21, about 15, about 8, or about 3 days or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders. 0-6 indicates normal/symptom absent; 7-19 indicates mild depression; 20-34 indicates moderate depression; and >34 indicates severe depression. In some embodiments, the therapeutic effect is a decrease from baseline in MADRS score at the end of a treatment period (e.g., about 45, about 21, about 15, about 8, or about 3 days or less). In some embodiments, the decrease from baseline in MADRS score is from severe (e.g., MADRS score of 30 or greater) to symptom-free (e.g., MADRS score of 20 or lower). For example, the mean change from baseline in MADRS total score from treatment with Compound (1) is about −15, −20, −25, −30, while the mean change from baseline in MADRS total score from treatment with placebo is about −15, −10, −5.

In some embodiments, the method provides therapeutic effect (e.g., as measured by reduction in Clinical Global Impression-Improvement Scale (CGI)) within about 45, about 21, about 15, about 8, or about 3 days or less. In some embodiments, the therapeutic effect is a CGI score of 2 or less.

In some embodiments, the method provides therapeutic effect (e.g., as measured by reduction in Edinburgh Postnatal Depression Scale (EPDS)) within 4, 3, 2, or 1 days; or 24, 20, 16, 12, 10, or 8 hours or less. In some embodiments, the therapeutic effect is an improvement measured by the EPDS.

In some embodiments, PPD with elevated anxiety is characterized by a Hamilton Rating Scale for Anxiety (HAM-A) total score of 17 or greater, 18 or greater, 19 or greater, or 20 or greater, or by a Hamilton Rating Scale for Depression (HAM-D) Anxiety/Somatization subscale score of 7 or greater, prior to the administration of Compound (1), or the pharmaceutically acceptable salt of Compound (1). In some embodiments, PPD with elevated anxiety is characterized by a Hamilton Rating Scale for Anxiety (HAM-A) total score of 17 or greater. In some embodiments, PPD with elevated anxiety is characterized by a Hamilton Rating Scale for Anxiety (HAM-A) total score of 18 or greater. In some embodiments, PPD with elevated anxiety is characterized by a Hamilton Rating Scale for Anxiety (HAM-A) total score of 19 or greater. In some embodiments, PPD with elevated anxiety is characterized by a Hamilton Rating Scale for Anxiety (HAM-A) total score of 20 or greater. In some embodiments, PPD with elevated anxiety is characterized by a Hamilton Rating Scale for Depression (HAM-D) Anxiety/Somatization subscale score of 7 or greater.

In some embodiments, PPD with elevated anxiety is characterized by a HAM-D total score of 24 or greater and a HAM-A total score of 17 or greater prior to the administration of Compound (1), or the pharmaceutically acceptable salt of Compound (1). In some embodiments, PPD with elevated anxiety is characterized by a HAM-D total score of 24 or greater and a HAM-A total score of 18 or greater prior to the administration of Compound (1), or the pharmaceutically acceptable salt of Compound (1). In some embodiments, PPD with elevated anxiety is characterized by a HAM-D total score of 24 or greater and a HAM-A total score of 19 or greater prior to the administration of Compound (1), or the pharmaceutically acceptable salt of Compound (1). In some embodiments, PPD with elevated anxiety is characterized by a HAM-D total score of 24 or greater and a HAM-A total score of 20 or greater prior to the administration of Compound (1), or the pharmaceutically acceptable salt of Compound (1). In some embodiments, PPD with elevated anxiety is characterized by a HAM-D total score of 24 or greater and a HAM-D Anxiety/Somatization subscale score of 7 or greater prior to the administration of Compound (1), or the pharmaceutically acceptable salt of Compound (1).

In some embodiments, PPD with elevated anxiety is characterized by a HAM-D total score of 26 or greater and a HAM-A total score of 17 or greater prior to the administration of Compound (1), or the pharmaceutically acceptable salt of Compound (1). In some embodiments, PPD with elevated anxiety is characterized by a HAM-D total score of 26 or greater and a HAM-A total score of 18 or greater prior to the administration of Compound (1), or the pharmaceutically acceptable salt of Compound (1). In some embodiments, PPD with elevated anxiety is characterized by a HAM-D total score of 26 or greater and a HAM-A total score of 19 or greater prior to the administration of Compound (1), or the pharmaceutically acceptable salt of Compound (1). In some embodiments, PPD with elevated anxiety is characterized by a HAM-D total score of 26 or greater and a HAM-A total score of 20 or greater prior to the administration of Compound (1), or the pharmaceutically acceptable salt of Compound (1). In some embodiments, PPD with elevated anxiety is characterized by a HAM-D total score of 26 or greater and a HAM-D Anxiety/Somatization subscale score of 7 or greater prior to the administration of Compound (1), or the pharmaceutically acceptable salt of Compound (1).

In other embodiments, “elevated anxiety” is characterized by a HAM-A score based on the HAM-A anxiety items and somatic items. In some embodiments, “elevated anxiety” is characterized by a HAM-A score based on the HAM-A anxiety items. In some embodiments, “elevated anxiety” is characterized by a HAM-D score based on the following HAM-D items: psychic anxiety, somatic anxiety, GI somatic symptoms, and/or general somatic symptoms. In some embodiments, “elevated anxiety” is characterized by a HAM-D score based on the following HAM-D item: psychic anxiety. In some embodiments, “elevated anxiety” is characterized by a HAM-D score based predominately on the items evaluating somatic symptoms of depression. In some embodiments, “elevated anxiety” is characterized by a HAM-D score based predominately on the items evaluating anxiety symptoms of depression. In some embodiments, “elevated anxiety” is characterized by a HAM-D Anxiety/Somatization Subscale score based predominately on the items evaluating somatic symptoms of depression. In some embodiments, “elevated anxiety” is characterized by a HAM-D Anxiety/Somatization Subscale score based predominately on the items evaluating anxiety symptoms of depression. In some embodiments, “elevated anxiety” is characterized by a MADRS score based predominately on the items evaluating the somatic symptoms of depression. In some embodiments, “elevated anxiety” is characterized by a MADRS score based predominately on the items evaluating the anxiety symptoms of depression.

### Major Depressive Disorder (MDD)

In another aspect, the present disclosure is directed to methods of treating major depressive disorder (MDD) in a human female subject during the subject's postnatal period. In some embodiments, the MDD is MDD with elevated anxiety.

The diagnosis and severity of the major depressive disorder treated by the methods described herein can be characterized as defined by the Diagnostic and Statistical Manual of Mental Disorders, 5^{th} Edition (DSM-5).

### Depressive Disorders

Depressive disorders include disruptive mood dysregulation disorder, major depressive disorder (including major depressive episode), persistent depressive disorder (dysthymia), premenstrual dysphoric disorder, substance/medication-induced depressive disorder, depressive disorder due to another medical condition, other specified depressive disorder, and unspecified depressive disorder. The common feature of all of these disorders is the presence of sad, empty, or irritable mood, accompanied by somatic and cognitive changes that significantly affect the individual's capacity to function. What differs among them are issues of duration, timing, or presumed etiology.

Major depressive disorder represents the classic condition in this group of disorders. It is characterized by discrete episodes of at least 2 weeks' duration (although most episodes last considerably longer) involving clear-cut changes in affect, cognition, and neurovegetative functions and inter-episode remissions. A discrete episode of major depressive disorder may be referred to as a “major depressive episode” or “depressive episode”.

### Major Depressive Disorder (MDD)

Major depressive disorder is generally known in the art.

In some embodiments, MDD is also known as depression or clinical depression and it is a mood disorder that causes a persistent feeling of sadness and loss of interest. MDD affects how a subject may feel, think, and behave, and can lead to a variety of emotional and physical problems.

In some embodiments, MDD is defined and diagnosed according to the DSM-5, for example, MDD is diagnosed according to Criterion A, as described below.

Criterion A. Five (or more) of the following symptoms have been present during the same 2-week period and represent a change from previous functioning; at least one of the symptoms is either (1) depressed mood or (2) loss of interest or pleasure.
1. Depressed mood most of the day, nearly every day, as indicated by either subjective report (e.g., feels sad, empty, hopeless) or observation made by others (e.g., appears tearful). (Note: In children and adolescents, can be irritable mood.)
2. Markedly diminished interest or pleasure in all, or almost all, activities most of the day, nearly every day (as indicated by either subjective account or observation).
3. Significant weight loss when not dieting or weight gain (e.g., a change of more than 5% of body weight in a month), or decrease or increase in appetite nearly every day (Note: In children, consider failure to make expected weight gain.)
4. Insomnia or hypersomnia nearly every day.
5. Psychomotor agitation or retardation nearly every day (observable by others, not merely subjective feelings of restlessness or being slowed down).
6. Fatigue or loss of energy nearly every day.
7. Feelings of worthlessness or excessive or inappropriate guilt (which may be delusional) nearly every day (not merely self-reproach or guilt about being sick).
8. Diminished ability to think or concentrate, or indecisiveness, nearly every day (either by subjective account or as observed by others).
9. Recurrent thoughts of death (not just fear of dying), recurrent suicidal ideation without a specific plan, or a suicide attempt or a specific plan for committing suicide.

Criteria B-E, described below, are additional descriptions of MDD and may be considered for describing or diagnosing MDD, but are not required.

Criterion B. The symptoms cause clinically significant distress or impairment in social, occupational, or other important areas of functioning.

Criterion C. The episode is not attributable to the physiological effects of a substance or to another medical condition.

Criteria A-C can represent a major depressive episode.

Criterion D. The occurrence of the major depressive episode is not better explained by schizoaffective disorder, schizophrenia, schizophreniform disorder, delusional disorder, or other specified and unspecified schizophrenia spectrum and other psychotic disorders.

Criterion E. There has never been a manic episode or a hypomanic episode.

In some embodiments, a major depressive episode (MDE) is a period characterized by the symptoms of MDD as described above.

In some embodiments, MDD is a clinical course that is characterized by one or more major depressive episodes (MDE) in a subject.

In some embodiments, MDD is diagnosed according to Criteria A-C, as described above. In some embodiments, MDD is diagnosed according to Criteria A-E, as described above.

### Diagnostic Features

The criterion symptoms for major depressive disorder must be present nearly every day to be considered present, with the exception of weight change and suicidal ideation. Depressed mood must be present for most of the day, in addition to being present nearly every day. Often insomnia or fatigue is the presenting complaint, and failure to probe for accompanying depressive symptoms will result in under diagnosis. Sadness may be denied at first but may be elicited through interview or inferred from facial expression and demeanor. With individuals who focus on a somatic complaint, clinicians should determine whether the distress from that complaint is associated with specific depressive symptoms. Fatigue and sleep disturbance are present in a high proportion of cases; psychomotor disturbances are much less common but are indicative of greater overall severity, as is the presence of delusional or near-delusional guilt.

The essential feature of a major depressive episode is a period of at least 2 weeks during which there is either depressed mood or the loss of interest or pleasure in nearly all activities (Criterion A above). In children and adolescents, the mood may be irritable rather than sad. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation or suicide plans or attempts. To count toward a major depressive episode, a symptom must either be newly present or must have clearly worsened compared with the person's pre-episode status. The symptoms must persist for most of the day, nearly every day, for at least 2 consecutive weeks. The episode must be accompanied by clinically significant distress or impairment in social, occupational, or other important areas of functioning. For some individuals with mild episodes, functioning may appear to be normal but requires markedly increased effort.

Sleep disturbance may take the form of either difficulty sleeping or sleeping excessively (Criterion A4). When insomnia is present, it typically takes the form of middle insomnia (e.g., waking up during the night and then having difficulty returning to sleep) or terminal insomnia (e.g., waking too early and being unable to return to sleep). Initial insomnia (e.g., difficulty falling asleep) may also occur. Individuals who present with over-sleeping (hypersomnia) may experience prolonged sleep episodes at night or increased daytime sleep. Sometimes the reason that the individual seeks treatment is for the disturbed sleep.

Major Depressive Disorder with Elevated Anxiety/Anxious Distress

The “anxious distress” identifier for MDD, as defined by the DSM-5, indicates the presence of at least two of the following symptoms during the majority of days of a major depressive episode or persistent depressive disorder (dysthymia):
1. Feeling keyed up or tense.
2. Feeling unusually restless.
3. Difficulty concentrating because of worry.
4. Fear that something awful may happen.
5. Feeling that the individual might lose control of himself or herself.

Severity is defined as:
Mild: Two symptoms.
Moderate: Three symptoms.
Moderate-severe: Four or five symptoms.
Severe: Four or five symptoms and with motor agitation.

Anxious distress has been noted as a prominent feature of both bipolar and major depressive disorder in both primary care and specialty mental health settings. High levels of anxiety have been associated with higher suicide risk, longer duration of illness, and greater likelihood of treatment nonresponse.

Accordingly, one aspect of the present disclosure is directed to a method of treating major depressive disorder (MDD) in a human female subject, the method comprising administering to the subject a therapeutically effective amount of Compound (1):

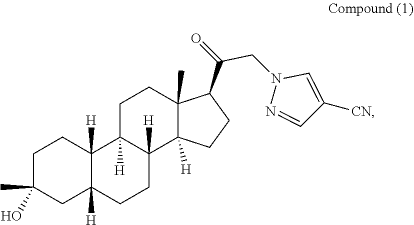

for a treatment period, wherein the subject breastfeeds a child during the treatment period.

Another aspect of the disclosure provides a method of treating major depressive disorder (MDD) in a human female subject, the method comprising administering to the subject a therapeutically effective amount a pharmaceutically acceptable salt of Compound (1):

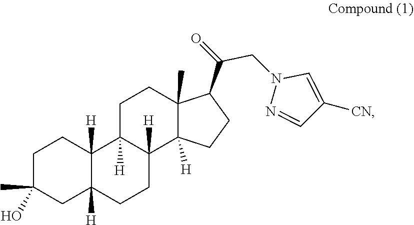

for a treatment period, wherein the subject breastfeeds a child during the treatment period.

Another aspect of the disclosure provides a method of treating major depressive disorder (MDD) with elevated anxiety in a human female subject, the method comprising administering to the subject a therapeutically effective amount of Compound (1):

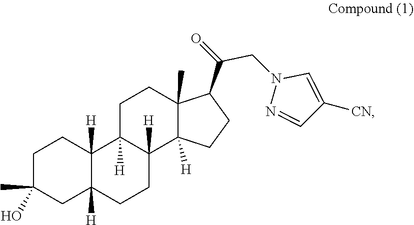

for a treatment period, wherein the subject breastfeeds a child during the treatment period.

Another aspect of the disclosure provides a method of treating major depressive disorder (MDD) with elevated anxiety in a human female subject, the method comprising administering to the subject a therapeutically effective amount a pharmaceutically acceptable salt of Compound (1):

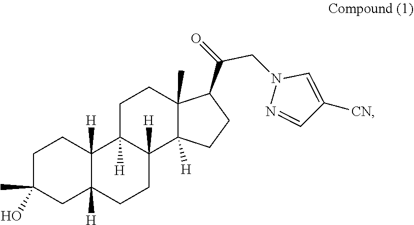

for a treatment period, wherein the subject breastfeeds a child during the treatment period.

In some embodiments, the subject breastfeeds the infant at least 1 time per day. In another embodiment, the subject breastfeeds the infant at least 2 times per day. In another embodiment, the subject breastfeeds the infant at least 3 times per day. In another embodiment, the subject breastfeeds the infant at least 4, 5, 6, 7, 8, 9, 10, 11, or 12 times per day.

In some embodiments, the treatment period is about 2 weeks or about 14 days. In some embodiments, the treatment period is about 2 weeks. In some embodiments, the treatment period is about 14 days.

In some embodiments, Compound (1), or the pharmaceutically acceptable salt of Compound (1), is administered once a day for about 14 days or about 2 weeks. In some embodiments, Compound (1), or the pharmaceutically acceptable salt of Compound (1), is administered once a day for about 14 days. In some embodiments, Compound (1), or the pharmaceutically acceptable salt of Compound (1), is administered once a day for about 2 weeks.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg to about 55 mg. In some embodiments, Compound (1) is administered at a dose of about 30 mg to about 50 mg. In some embodiments, Compound (1) is administered at a dose of about 45 mg to about 55 mg. In some embodiments, Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg. In some embodiments, Compound (1) is administered at a dose of about 30 mg.

In some embodiments, Compound (1) is administered at a dose of about 10 mg to about 100 mg once a day. In some embodiments, Compound (1) is administered at a dose of about 15 mg to about 75 mg once a day. In some embodiments, Compound (1) is administered at a dose of about 20 mg to about 60 mg once a day. In some embodiments, Compound (1) is administered at a dose of about 20 mg to about 55 mg once a day. In some embodiments, Compound (1) is administered at a dose of about 30 mg to about 50 mg once a day. In some embodiments, Compound (1) is administered at a dose of about 45 mg to about 55 mg once a day. In some embodiments, Compound (1) is administered at a dose of about 20 mg, about 25 mg, about 30 mg, about 35 mg, about 40 mg, about 45 mg, about 50 mg, about 55 mg, or about 60 mg once a day. In some embodiments, Compound (1) is administered at a dose of about 50 mg once a day. In some embodiments, Compound (1) is administered at a dose of about 40 mg once a day. In some embodiments, Compound (1) is administered at a dose of about 30 mg once a day.

In some embodiments, Compound (1) is administered at a dose of about 20 mg to about 55 mg once a day for about 2 weeks or about 14 days. In some embodiments, Compound (1) is administered at a dose of about 30 mg to about 50 mg once a day for about 2 weeks or about 14 days. In some embodiments, Compound (1) is administered at a dose of about 45 mg to about 55 mg once a day for about 2 weeks or about 14 days. In some embodiments, Compound (1) is administered at a dose of about 50 mg once a day for less than 2 weeks. In some embodiments, Compound (1) is administered at a dose of about 50 mg once a day for about 2 weeks. In some embodiments, Compound (1) is administered at a dose of about 50 mg once a day for about 14 days. In some embodiments, Compound (1) is administered at a dose of about 40 mg once a day for less than 2 weeks. In some embodiments, Compound (1) is administered at a dose of about 40 mg once a day for about 2 weeks. In some embodiments, Compound (1) is administered at a dose of about 40 mg once a day for about 14 days. In some embodiments, Compound (1) is administered at a dose of about 30 mg once a day for less than 2 weeks. In some embodiments, Compound (1) is administered at a dose of about 30 mg once a day for about 2 weeks. In some embodiments, Compound (1) is administered at a dose of about 30 mg once a day for about 14 days.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 45 mg to about 55 mg of the free base compound. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound. In some embodiments, the pharmaceutically acceptable salt of Compound (1) is administered at a dose equivalent of about 30 mg of the free base compound.

In some embodiments, the pharmaceutically acceptable salt of Compound (1) is administered at a dose equivalent of about 10 mg to about 100 mg of the free base compound once a day. In some embodiments, the pharmaceutically acceptable salt of Compound (1) is administered at a dose equivalent of about 15 mg to about 75 mg of the free base compound once a day. In some embodiments, the pharmaceutically acceptable salt of Compound (1) is administered at a dose equivalent of about 20 mg to about 60 mg of the free base compound once a day. In some embodiments, the pharmaceutically acceptable salt of Compound (1) is administered at a dose equivalent of about 20 mg to about 55 mg of the free base compound once a day. In some embodiments, the pharmaceutically acceptable salt of Compound (1) is administered at a dose equivalent of about 30 mg to about 50 mg of the free base compound once a day. In some embodiments, the pharmaceutically acceptable salt of Compound (1) is administered at a dose equivalent of about 45 mg to about 55 mg of the free base compound once a day. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) is administered at a dose equivalent of about 50 mg of the free base compound once a day. In some embodiments, the pharmaceutically acceptable salt of Compound (1) is administered at a dose equivalent of about 40 mg of the free base compound once a day. In some embodiments, the pharmaceutically acceptable salt of Compound (1) is administered at a dose equivalent of about 30 mg of the free base compound once a day.

In some embodiments, the pharmaceutically acceptable salt of Compound (1) is administered at a dose equivalent of about 20 mg to about 55 mg of the free base compound once a day for about 2 weeks or about 14 days. In some embodiments, the pharmaceutically acceptable salt of Compound (1) is administered at a dose equivalent of about 30 mg to about 50 mg of the free base compound once a day for about 2 weeks or about 14 days. In some embodiments, the pharmaceutically acceptable salt of Compound (1) is administered at a dose equivalent of about 45 mg to about 55 mg of the free base compound once a day for about 2 weeks or about 14 days. In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 2 weeks. In some embodiments, the pharmaceutically acceptable salt of Compound (1) is administered at a dose equivalent of about 50 mg of the free base compound once a day for about 14 days. In some embodiments, the pharmaceutically acceptable salt of Compound (1) is administered at a dose equivalent of about 40 mg of the free base compound once a day for less than 2 weeks. In some embodiments, the pharmaceutically acceptable salt of Compound (1) is administered at a dose equivalent of about 40 mg of the free base compound once a day for about 2 weeks. In some embodiments, the pharmaceutically acceptable salt of Compound (1) is administered at a dose equivalent of about 40 mg of the free base compound once a day for about 14 days. In some embodiments, the pharmaceutically acceptable salt of Compound (1) is administered at a dose equivalent of about 30 mg of the free base compound once a day for less than 2 weeks. In some embodiments, the pharmaceutically acceptable salt of Compound (1) is administered at a dose equivalent of about 30 mg of the free base compound once a day for about 2 weeks. In some embodiments, the pharmaceutically acceptable salt of Compound (1) is administered at a dose equivalent of about 30 mg of the free base compound once a day for about 14 days.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered chronically.

In some embodiments, Compound (1), or the pharmaceutically acceptable salt of Compound (1), is administered in one or more capsules. In some embodiments, the therapeutically effective amount is administered across two capsules. In some embodiments, the therapeutically effective amount is administered across three capsules.

In some embodiments, Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps. In some embodiments, the subject is administered Compound (1), or the pharmaceutically acceptable salt of Compound (1), once a day at night. In some embodiments, the subject is administered Compound (1), or the pharmaceutically acceptable salt of Compound (1), once a day no later than 1 hour before the patient sleeps. In some embodiments, the subject is administered Compound (1), or the pharmaceutically acceptable salt of Compound (1), once a day no later than 15 minutes before the patient sleeps.

In some embodiments, Compound (1) is in a crystalline form. In some embodiments, the crystalline form of Compound (1) is any crystalline form disclosed in PCT Application Publication No. WO 2018/039378; the entire contents of the aforementioned application are incorporated herein by reference in its entirety.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 20. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 20. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the crystalline form of Compound (1) comprises a mixture of two or more crystalline forms.

In some embodiments, the subject is treatment naïve. In some embodiments, the subject has not received any antidepressant treatment within at least 30 days prior to the administration of Compound (1), or the pharmaceutically acceptable salt of Compound (1). In some embodiments, the subject has not received any antidepressant treatment within at least 60 days prior to the administration of Compound (1), or the pharmaceutically acceptable salt of Compound (1).

In some embodiments, the subject has been on a stable dose of an additional antidepressant for at least 30 days or for at least 60 days prior to the beginning of the treatment period. In some embodiments, the subject has been on a stable dose of an additional antidepressant for at least 60 days prior to the beginning of the treatment period. In some embodiments, the subject has been on a stable dose of an additional antidepressant for at least 30 days prior to the beginning of the treatment period.

In some embodiments, the breast milk of the subject is monitored to determine relative infant dose (RID) of Compound (1), or the pharmaceutically acceptable salt of Compound (1), in the breast milk, and the daily dose of Compound (1) is adjusted to produce less than a maximum relative infant dose (RID). RID estimates infant drug exposure via breast milk. The RID uses a known milk concentration and compares it to either an infant therapeutic dose or the weight-adjusted maternal dose when an infant dose is not well established. Typically, breastfeeding is considered acceptable when the relative infant dose is <10%. Additional considerations include the gestational and postnatal age of the infant, the actual amount of milk being ingested (less in the first couple days of life and when weaning), properties of the specific maternal medication, medical conditions of the infant, and medications the infant is receiving therapeutically.

In some embodiments, the maximum relative infant dose (RID) is at most about 0.5% of the daily dose administered to the subject. In some embodiments, the maximum relative infant dose (RID) is at most about 0.4% of the daily dose administered to the subject. In some embodiments, the maximum relative infant dose (RID) is at most about 0.357% of the daily dose administered to the subject.

In some embodiments, the child is monitored for abnormal behavior. In some embodiments, the abnormal behavior is selected from the group consisting of agitation, irritability, lethargy, oversleeping, poor feeding, and poor weight gain.

In some embodiments, the daily dose administered to the subject is decreased by 10 mg if the relative infant dose is above the maximum relative infant dose (RID), or if the child shows abnormal behavior. In some embodiments, the daily dose administered to the subject is decreased by 10 mg if the relative infant dose is above the maximum relative infant dose (RID). In some embodiments, the daily dose administered to the subject is decreased by 10 mg if the child shows abnormal behavior.

In some embodiments, the method further comprises administration of a second therapeutic agent.

In some embodiments, Compound (1), or the pharmaceutically acceptable salt of Compound (1), is re-administered to the subject in response to a recurrence of depression symptoms after completion of initial treatment period. In some embodiments, there is at least a 6 week interval between the last dose of the initial treatment period and the first dose of the re-administration. In some embodiments, each of the initial treatment period and re-administration occurs for about 14 days or about 2 weeks.

In some embodiments, MDD with elevated anxiety is characterized by a Hamilton Rating Scale for Anxiety (HAM-A) total score of 17 or greater, 18 or greater, 19 or greater, or 20 or greater. In some embodiments, MDD with elevated anxiety is characterized by a Hamilton Rating Scale for Anxiety (HAM-A) total score of 17 or greater, or by a Hamilton Rating Scale for Depression (HAM-D) Anxiety/Somatization subscale score of 7 or greater, prior to the administration of Compound (1), or the pharmaceutically acceptable salt of Compound (1). In some embodiments, MDD with elevated anxiety is characterized by a Hamilton Rating Scale for Anxiety (HAM-A) total score of 17 or greater. In some embodiments, MDD with elevated anxiety is characterized by a Hamilton Rating Scale for Anxiety (HAM-A) total score of 18 or greater. In some embodiments, MDD with elevated anxiety is characterized by a Hamilton Rating Scale for Anxiety (HAM-A) total score of 19 or greater. In some embodiments, MDD with elevated anxiety is characterized by a Hamilton Rating Scale for Anxiety (HAM-A) total score of 20 or greater. In some embodiments, MDD with elevated anxiety is characterized by a Hamilton Rating Scale for Depression (HAM-D) Anxiety/Somatization subscale score of 7 or greater.

In some embodiments, MDD with elevated anxiety is characterized by a HAM-D total score of 24 or greater and a HAM-A total score of 17 or greater prior to the administration of Compound (1), or the pharmaceutically acceptable salt of Compound (1). In some embodiments, MDD with elevated anxiety is characterized by a HAM-D total score of 24 or greater and a HAM-A total score of 18 or greater prior to the administration of Compound (1), or the pharmaceutically acceptable salt of Compound (1). In some embodiments, MDD with elevated anxiety is characterized by a HAM-D total score of 24 or greater and a HAM-A total score of 19 or greater prior to the administration of Compound (1), or the pharmaceutically acceptable salt of Compound (1). In some embodiments, MDD with elevated anxiety is characterized by a HAM-D total score of 24 or greater and a HAM-A total score of 20 or greater prior to the administration of Compound (1), or the pharmaceutically acceptable salt of Compound (1). In some embodiments, MDD with elevated anxiety is characterized by a HAM-D total score of 24 or greater and a HAM-D Anxiety/Somatization subscale score of 7 or greater prior to the administration of Compound (1), or the pharmaceutically acceptable salt of Compound (1).

In some embodiments, MDD with elevated anxiety is characterized by a HAM-D total score of 20 or greater, a MADRS total score of 28 or greater, and a HAM-A total score of 17 or greater (e.g., 18 or greater, 19 or greater, or 20 or greater) prior to the administration of Compound (1), or the pharmaceutically acceptable salt of Compound (1). In some embodiments, MDD with elevated anxiety is characterized by a HAM-D total score of 20 or greater, a MADRS total score of 28 or greater, and a HAM-D Anxiety/Somatization subscale score of 7 or greater prior to the administration of Compound (1), or the pharmaceutically acceptable salt of Compound (1).

In other embodiments, “elevated anxiety” is characterized by a HAM-A score based on the HAM-A anxiety items and somatic items. In some embodiments, “elevated anxiety” is characterized by a HAM-A score based on the HAM-A anxiety items. In some embodiments, “elevated anxiety” is characterized by a HAM-D score based on the following HAM-D items: psychic anxiety, somatic anxiety, GI somatic symptoms, and/or general somatic symptoms. In some embodiments, “elevated anxiety” is characterized by a HAM-D score based on the following HAM-D item: psychic anxiety. In some embodiments, “elevated anxiety” is characterized by a HAM-D score based predominately on the items evaluating somatic symptoms of depression. In some embodiments, “elevated anxiety” is characterized by a HAM-D score based predominately on the items evaluating anxiety symptoms of depression. In some embodiments, “elevated anxiety” is characterized by a HAM-D Anxiety/Somatization Subscale score based predominately on the items evaluating somatic symptoms of depression. In some embodiments, “elevated anxiety” is characterized by a HAM-D Anxiety/Somatization Subscale score based predominately on the items evaluating anxiety symptoms of depression. In some embodiments, “elevated anxiety” is characterized by a MADRS score based predominately on the items evaluating the somatic symptoms of depression. In some embodiments, “elevated anxiety” is characterized by a MADRS score based predominately on the items evaluating the anxiety symptoms of depression.

Another aspect of the disclosure provides a method of treating postpartum depression (PPD) in a human female subject during the subject's postnatal period, the method comprising:
a) administering to the subject about 30 mg to about 50 mg of Compound (1) once a day for about 14 days:

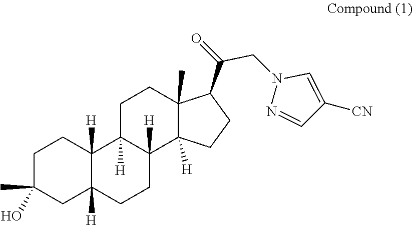

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

Another aspect of the disclosure provides a method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days:

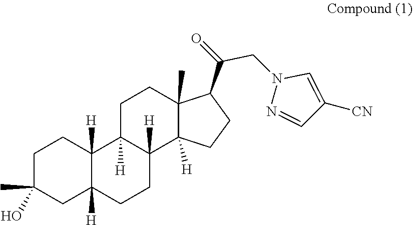

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

Another aspect of the disclosure provides a method of treating major depressive disorder (MDD) in a human female subject, the method comprising:
a) administering to the subject about 30 mg to about 50 mg of Compound (1) once a day for about 14 days:

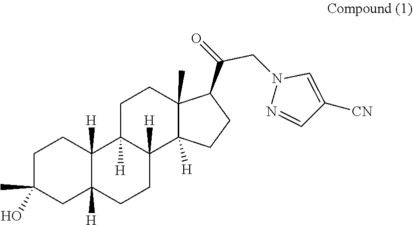

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

Another aspect of the disclosure provides a method of treating major depressive disorder (MDD) in a human female, the method comprising:
a) administering to the subject a pharmaceutically acceptable salt of Compound (1) at a dose equivalent to about 30 mg to about 50 mg of the free base compound once a day for about 14 days:

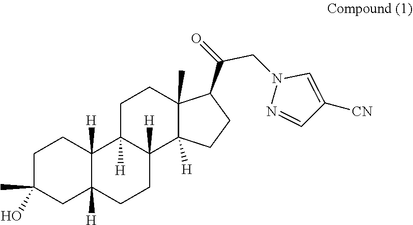

for a treatment period, wherein the subject breastfeeds a child during the treatment period;
b) comparing the relative infant dose of Compound (1) in the subject's breast milk with a predetermined maximum relative infant dose; and
c) lowering the daily dose administered to the subject if the relative infant dose of Compound (1) in the subject's breast milk is above the predetermined maximum relative infant dose, or if the child is exhibiting abnormal behavior.

Another aspect of the disclosure provides a method of treating postpartum depression (PPD) in a human female subject during the subject's postnatal period, the method comprising:
a) administering to the subject about 30 mg to about 50 mg of Compound (1) once a day for about 14 days:

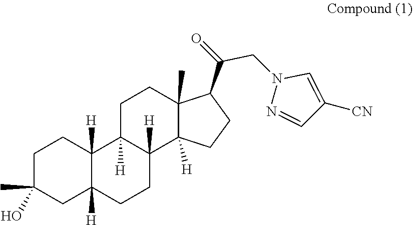

for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

Another aspect of the disclosure provides a method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days:

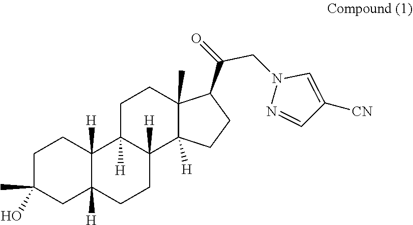

for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

Another aspect of the disclosure provides a method of treating major depressive disorder (MDD) in a human female subject, the method comprising:
a) administering to the subject about 30 mg to about 50 mg of Compound (1) once a day for about 14 days:

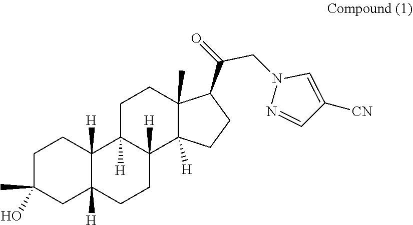

for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

Another aspect of the disclosure provides a method of treating major depressive disorder (MDD) in a human female subject, the method comprising:
a) administering to the subject a pharmaceutically acceptable salt of Compound (1) at a dose equivalent to about 30 mg to about 50 mg of the free base compound once a day for about 14 days:

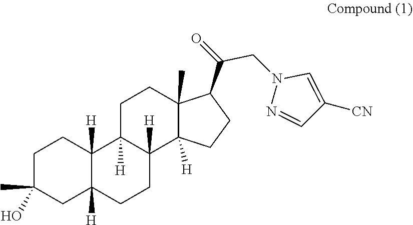

for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

In some embodiments, the PPD is PPD with elevated anxiety. In some embodiments, the MDD is MDD with elevated anxiety.

In some embodiments, MDD with elevated anxiety or PPD with elevated anxiety is characterized by a Hamilton Rating Scale for Anxiety (HAM-A) total score of 17 or greater, 18 or greater, 19 or greater, or 20 or greater, or by a Hamilton Rating Scale for Depression (HAM-D) Anxiety/Somatization subscale score of 7 or greater, prior to the administration of Compound (1), or the pharmaceutically acceptable salt of Compound (1). In some embodiments, MDD with elevated anxiety or PPD with elevated anxiety is characterized by a Hamilton Rating Scale for Anxiety (HAM-A) total score of 17 or greater prior to the administration of Compound (1), or the pharmaceutically acceptable salt of Compound (1). In some embodiments, MDD with elevated anxiety or PPD with elevated anxiety is characterized by a Hamilton Rating Scale for Anxiety (HAM-A) total score of 18 or greater prior to the administration of Compound (1), or the pharmaceutically acceptable salt of Compound (1). In some embodiments, MDD with elevated anxiety or PPD with elevated anxiety is characterized by a Hamilton Rating Scale for Anxiety (HAM-A) total score of 19 or greater prior to the administration of Compound (1), or the pharmaceutically acceptable salt of Compound (1). In some embodiments, MDD with elevated anxiety or PPD with elevated anxiety is characterized by a Hamilton Rating Scale for Anxiety (HAM-A) total score of 20 or greater prior to the administration of Compound (1), or the pharmaceutically acceptable salt of Compound (1). In some embodiments, MDD with elevated anxiety or PPD with elevated anxiety is characterized by a Hamilton Rating Scale for Depression (HAM-D) Anxiety/Somatization subscale score of 7 or greater, prior to the administration of Compound (1), or the pharmaceutically acceptable salt of Compound (1).

In some embodiments, MDD with elevated anxiety or PPD with elevated anxiety is characterized by a HAM-D total score of 24 or greater and a HAM-A total score of 17 or greater prior to the administration of Compound (1), or the pharmaceutically acceptable salt of Compound (1). In some embodiments, MDD with elevated anxiety or PPD with elevated anxiety is characterized by a HAM-D total score of 24 or greater and a HAM-A total score of 18 or greater prior to the administration of Compound (1), or the pharmaceutically acceptable salt of Compound (1). In some embodiments, MDD with elevated anxiety or PPD with elevated anxiety is characterized by a HAM-D total score of 24 or greater and a HAM-A total score of 19 or greater prior to the administration of Compound (1), or the pharmaceutically acceptable salt of Compound (1). In some embodiments, MDD with elevated anxiety or PPD with elevated anxiety is characterized by a HAM-D total score of 24 or greater and a HAM-A total score of 20 or greater prior to the administration of Compound (1), or the pharmaceutically acceptable salt of Compound (1).

In some embodiments, MDD with elevated anxiety or PPD with elevated anxiety is characterized by a HAM-D total score of 24 or greater and a HAM-D Anxiety/Somatization subscale score of 7 or greater prior to the administration of Compound (1), or the pharmaceutically acceptable salt of Compound (1).

In some embodiments, MDD with elevated anxiety or PPD with elevated anxiety is characterized by a HAM-D total score of 26 or greater and a HAM-A total score of 17 or greater (e.g., 18 or greater, 19 or greater, or 20 or greater) prior to the administration of Compound (1), or the pharmaceutically acceptable salt of Compound (1). In some embodiments, MDD with elevated anxiety or PPD with elevated anxiety is characterized by a HAM-D total score of 26 or greater and a HAM-D Anxiety/Somatization subscale score of 7 or greater prior to the administration of Compound (1), or the pharmaceutically acceptable salt of Compound (1).

In some embodiments, MDD with elevated anxiety or PPD with elevated anxiety is characterized by a HAM-D total score of 20 or greater, a MADRS total score of 28 or greater, and a HAM-A total score of 17 or greater (e.g., 18 or greater, 19 or greater, or 20 or greater) prior to the administration of Compound (1), or the pharmaceutically acceptable salt of Compound (1). In some embodiments, MDD with elevated anxiety or PPD with elevated anxiety is characterized by a HAM-D total score of 20 or greater, a MADRS total score of 28 or greater, and a HAM-D Anxiety/Somatization subscale score of 7 or greater prior to the administration of Compound (1), or the pharmaceutically acceptable salt of Compound (1).

In other embodiments, “elevated anxiety” is characterized by a HAM-A score based on the HAM-A anxiety items and somatic items. In some embodiments, “elevated anxiety” is characterized by a HAM-A score based on the HAM-A anxiety items. In some embodiments, “elevated anxiety” is characterized by a HAM-D score based on the following HAM-D items: psychic anxiety, somatic anxiety, GI somatic symptoms, and general somatic symptoms. In some embodiments, “elevated anxiety” is characterized by a HAM-D score based on the following HAM-D item: psychic anxiety. In some embodiments, “elevated anxiety” is characterized by a HAM-D score based predominately on the items evaluating somatic symptoms of depression. In some embodiments, “elevated anxiety” is characterized by a HAM-D score based predominately on the items evaluating anxiety symptoms of depression. In some embodiments, “elevated anxiety” is characterized by a HAM-D Anxiety/Somatization Subscale score based predominately on the items evaluating somatic symptoms of depression. In some embodiments, “elevated anxiety” is characterized by a HAM-D Anxiety/Somatization Subscale score based predominately on the items evaluating anxiety symptoms of depression. In some embodiments, “elevated anxiety” is characterized by a MADRS score based predominately on the items evaluating the somatic symptoms of depression. In some embodiments, “elevated anxiety” is characterized by a MADRS score based predominately on the items evaluating the anxiety symptoms of depression.

In some embodiments, Compound (1) is administered at a dose of about 50 mg or the pharmaceutically acceptable salt of Compound (1) is administered at a dose equivalent to about 50 mg of the free base compound. In some embodiments, Compound (1) is administered at a dose of about 40 mg or the pharmaceutically acceptable salt of Compound (1) is administered at a dose equivalent to about 40 mg of the free base compound. In some embodiments, Compound (1) is administered at a dose of about 30 mg or the pharmaceutically acceptable salt of Compound (1) is administered at a dose equivalent to about 30 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered chronically.

In some embodiments, Compound (1), or the pharmaceutically acceptable salt of Compound (1), is administered in one or more capsules. In some embodiments, the therapeutically effective amount is administered across two capsules. In some embodiments, the therapeutically effective amount is administered across three capsules.

In some embodiments, Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps. In some embodiments, the subject is administered Compound (1), or the pharmaceutically acceptable salt of Compound (1), once a day at night. In some embodiments, the subject is administered Compound (1), or the pharmaceutically acceptable salt of Compound (1), once a day no later than 1 hour before the patient sleeps. In some embodiments, the subject is administered Compound (1), or the pharmaceutically acceptable salt of Compound (1), once a day no later than 15 minutes before the patient sleeps.

In some embodiments, the subject is treatment naïve. In some embodiments, the subject has not received any antidepressant treatment within at least 30 days prior to the administration of Compound (1), or the pharmaceutically acceptable salt of Compound (1). In some embodiments, the subject has not received any antidepressant treatment within at least 60 days prior to the administration of Compound (1), or the pharmaceutically acceptable salt of Compound (1).

In some embodiments, the subject has been on a stable dose of an additional antidepressant for at least 30 days or for at least 60 days prior to the beginning of the treatment period. In some embodiments, the subject has been on a stable dose of an additional antidepressant for at least 60 days prior to the beginning of the treatment period. In some embodiments, the subject has been on a stable dose of an additional antidepressant for at least 30 days prior to the beginning of the treatment period.

In some embodiments, the method further comprises administration of a second therapeutic agent.

In some embodiments, Compound (1), or the pharmaceutically acceptable salt of Compound (1), is re-administered to the subject in response to a recurrence of depression symptoms after completion of the initial treatment period. In some embodiments, there is at least a 6 week interval between the last dose of the initial treatment period and the first dose of the re-administration. In some embodiments, each of the initial treatment period and re-administration occurs for about 14 days or about 2 weeks.

In some embodiments, the maximum relative infant dose (RID) is at most about 0.5% of the daily dose administered to the subject. In some embodiments, the maximum relative infant dose (RID) is at most about 0.4% of the daily dose administered to the subject. In some embodiments, the maximum relative infant dose (RID) is at most about 0.357% of the daily dose administered to the subject.

In some embodiments, the abnormal behavior is selected from the group consisting of agitation, irritability, lethargy, oversleeping, poor feeding, and poor weight gain.

### III. Pharmaceutical Compositions

Another aspect of the disclosure provides a pharmaceutical composition comprising Compound (1) (also referred to as the “active ingredient”), and a pharmaceutically acceptable excipient for use in the methods described herein. In another aspect, the disclosure provides a pharmaceutical composition comprising a pharmaceutically acceptable salt of the active ingredient and a pharmaceutically acceptable excipient for use in the methods described herein. In certain embodiments, the pharmaceutical composition comprises an effective amount of the active ingredient or a pharmaceutically acceptable salt of the active ingredient. In certain embodiments, the pharmaceutical composition comprises a therapeutically effective amount of the active ingredient or a pharmaceutically acceptable salt of the active ingredient. In some embodiments, the pharmaceutical composition of Compound (1) is any pharmaceutical composition disclosed in PCT Application Publication No. WO 2022/020363A9; the entire contents of the aforementioned application are incorporated herein by reference in its entirety.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical composition is administered orally.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials, as well as processing techniques and the like, are set forth in Part 8 of Remington's Pharmaceutical Sciences, 17th edition, 1985, Mack Publishing Company, Easton, Pennsylvania, which is incorporated herein by reference.

The compositions of the present disclosure can also be administered in sustained release forms or from sustained release drug delivery systems. A description of representative sustained release materials can be found in Remington's Pharmaceutical Sciences.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

The present disclosure also relates to the pharmaceutically acceptable acid addition salt of Compound (1). The acid which may be used to prepare the pharmaceutically acceptable salt is that which forms a non-toxic acid addition salt, e.g., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

Another aspect of the disclosure includes a method of treating postpartum depression in a human female subject during the subject's postnatal period, the method comprising administering to the subject a therapeutically effective amount of Compound (1), for a treatment period, wherein the subject breastfeeds an infant during the treatment period.

In embodiments of this aspect, the treatment period is about 14 days. In some embodiments, the therapeutically effective amount is administered once per day. In some embodiments, the subject breastfeeds the infant at least 3 times per day.

In some embodiments, the therapeutically effective amount is from about 10 mg to about 55 mg of Compound (1) per day. In some embodiments, the therapeutically effective amount is from about 20 mg to about 55 mg of Compound (1) per day. In some embodiments, the therapeutically effective amount is about 30 mg of Compound (1) per day. In some embodiments, the therapeutically effective amount is about 50 mg of Compound (1) per day.

In some embodiments, the breast milk of the subject is monitored to determine relative infant dose (RID) of Compound (1) in the breast milk, and the daily dose of Compound (1) is adjusted to produce less than a maximum RID. In some embodiments, the maximum RID is at most about 0.5% of the daily dose administered to the subject. In some embodiments, the maximum RID is at most about 0.4% of the daily dose administered to the subject. In some embodiments, the maximum RID is at most about 0.357% of the daily dose administered to the subject.

In some embodiments, the infant is monitored for abnormal behavior. In some embodiments, the abnormal behavior is selected from the group consisting of agitation, irritability, lethargy, oversleeping, poor feeding, and poor weight gain.

In some embodiments, the daily dose administered to the subject is decreased by 10 mg if the RID is above the maximum RID, or if the infant shows abnormal behavior.

Another aspect of the disclosure includes a method of treating postpartum depression in a human female subject during the subject's postnatal period, the method comprising:
a) administering to the subject a 30 mg or a 50 mg daily dose of Compound (1),

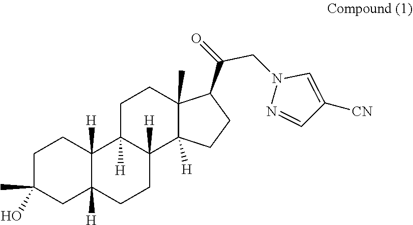

for a treatment period, wherein the subject breastfeeds an infant during the treatment period;
b) collecting and testing a sample of breast milk to determine the relative infant dose (RID) of Compound (1) in the breast milk;
c) comparing the RID determined in step b) with a predetermined maximum RID;
d) monitoring the infant for abnormal behavior;
e) lowering the daily dose administered to the subject if the RID determined in step b) is above the predetermined maximum RID, or if the infant is exhibiting abnormal behavior; and
f) repeating steps a)-e) each day for the duration of the treatment period.

In embodiments of this aspect, the daily dose is 50 mg. In some embodiments, the daily dose is 30 mg.

In some embodiments, the predetermined maximum RID is at least about 0.5% of the daily dose administered to the subject. In some embodiments, the maximum RID is at least about 0.4% of the daily dose administered to the subject. In some embodiments, the maximum RID is at least about 0.357% of the daily dose administered to the subject.

In some embodiments, the abnormal behavior for the infant is selected from the group consisting of agitation, irritability, lethargy, oversleeping, poor feeding, and poor weight gain.

### EXAMPLES

### Example 1. An Open-Label Study to Evaluate Concentrations of Compound (1) in the Breast Milk of Healthy Lactating Women

### Objective

Compound (1) is an investigational, synthetic, oral neuroactive steroid under investigation as a once-daily therapy for major depressive disorder and postpartum depression. This open-label study evaluated the extent of Compound (1) transfer into breast milk, breast milk production (volume), pharmacokinetics (PK), plasma protein binding, safety, and tolerability in healthy lactating participants.

### Methods

Healthy volunteers (N=15) aged 18-45 years, >12 weeks postpartum, actively lactating, and pumping or breast feeding ≥3 times per day orally self-administered Compound (1) 30 mg once-daily with food for 5 days. Breast milk was collected from Day −3 through Day 12. Blood samples for PK and plasma protein binding analysis were collected predose (Day 1), on Days 5-6, and during follow-up (Days 7, 9, and 12). The relationship between plasma and breast milk concentrations was examined with population PK modeling.

### Results

Fifteen participants received ≥1 dose of Compound (1) and 14 (93.3%) completed the study. Participants' PK profiles were similar to other clinical studies with Compound (1). In plasma, Compound (1) was highly protein bound, with a free fraction ≤0.52% across participants. Compared to maternal dose, breast milk amounts of Compound (1) were low, with an estimated mean relative infant dose (RID) of 0.357% and daily infant dose of 0.00125 mg/kg/day on Day 5. Volume of milk collected decreased 8.3% from baseline; inter-participant variability was noted.

Compound (1) concentrations were described with a constant partition coefficient of 0.501 between milk and plasma; between-subject variability in partitioning was 23%. Compound (1) plasma concentrations had no apparent relationship with time or collected milk volume.

No deaths, serious adverse events, moderate or severe treatment-emergent adverse events (TEAEs), or TEAEs leading to discontinuation or study withdrawal were reported.

### Conclusions

Compound (1) 30 mg well-tolerated when administered to healthy lactating participants for 5 days. Only a small amount was transferred to breast milk, resulting in an RID of 0.357%. Minimal concentrations were detected in breast milk (approximately one-half of plasma concentrations) with low between-subject variability in partitioning. Breastfeeding while taking Compound (1) may be considered appropriate if benefits outweigh risks to mother and baby.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A method of treating postpartum depression (PPD) in a human female subject during the subject's postnatal period, the method comprising administering to the subject a therapeutically effective amount of Compound (1): 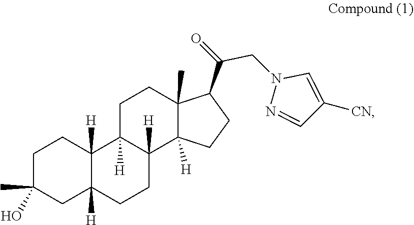
for a treatment period, wherein the subject breastfeeds a child during the treatment period.

2. A method of treating postpartum depression (PPD) in a human female subject during the subject's postnatal period, the method comprising administering to the subject a therapeutically effective amount a pharmaceutically acceptable salt of Compound (1): 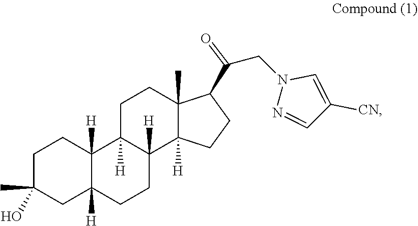
for a treatment period, wherein the subject breastfeeds a child during the treatment period.

3. A method of treating postpartum depression (PPD) with elevated anxiety in a human female subject during the subject's postnatal period, the method comprising administering to the subject a therapeutically effective amount of Compound (1): 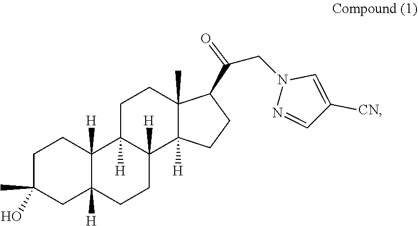
for a treatment period, wherein the subject breastfeeds a child during the treatment period.

4. A method of treating postpartum depression (PPD) with elevated anxiety in a human female subject during the subject's postnatal period, the method comprising administering to the subject a therapeutically effective amount a pharmaceutically acceptable salt of Compound (1): 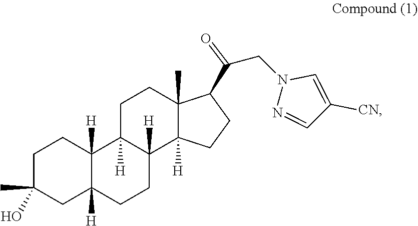
for a treatment period, wherein the subject breastfeeds a child during the treatment period.

5. The method according to any one of claims 1-4, wherein the subject breastfeeds the child at least 3 times per day.

6. The method according to any one of claims 1-4, wherein the treatment period is about 2 weeks or about 14 days.

7. The method according to any one of claims 1-4, wherein Compound (1), or the pharmaceutically acceptable salt of Compound (1), is administered once a day for about 14 days.

8. The method according to claim 1 or claim 3, wherein Compound (1) is administered at a dose of about 20 mg to about 55 mg.

9. The method according to claim 1 or claim 3, wherein Compound (1) is administered at a dose of about 50 mg.

10. The method according to claim 1 or claim 3, wherein Compound (1) is administered at a dose of about 40 mg.

11. The method according to claim 1 or claim 3, wherein Compound (1) is administered at a dose of about 30 mg.

12. The method according to claim 2 or claim 4, wherein the pharmaceutically acceptable salt of Compound (1) is administered at a dose equivalent to about 20 mg to about 55 mg of the free base compound.

13. The method according to claim 2 or claim 4, wherein the pharmaceutically acceptable salt of Compound (1) is administered at a dose equivalent to about 50 mg of the free base compound.

14. The method according to claim 2 or claim 4, wherein the pharmaceutically acceptable salt of Compound (1) is administered at a dose equivalent to about 40 mg of the free base compound.

15. The method according to claim 2 or claim 4, wherein the pharmaceutically acceptable salt of Compound (1) is administered at a dose equivalent to about 30 mg of the free base compound.

16. The method according to any one of claims 1-15, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasally, or transdermally.

17. The method according to claim 16, wherein Compound (1), or the pharmaceutically acceptable salt of Compound (1), is administered orally.

18. The method according to any one of claims 1-17, wherein Compound (1), or the pharmaceutically acceptable salt of Compound (1), is administered with food.

19. The method according to any one of claims 1-18, wherein Compound (1), or the pharmaceutically acceptable salt of Compound (1), is administered once a day at night.

20. The method according to claim 1 or claim 3, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

21. The method according to claim 1 or claim 3, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

22. The method according to claim 1 or claim 3, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

23. The method according to claim 1 or claim 3, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

24. The method according to any one of claims 1-23, wherein the subject is treatment naïve.

25. The method according to any one of claims 1-23, wherein the subject has been on a stable dose of an additional antidepressant for at least 30 days or for at least 60 days prior to the beginning of the treatment period.

26. The method according to any one of claims 1-25, wherein the breast milk of the subject is monitored to determine relative infant dose of Compound (1), or the pharmaceutically acceptable salt of Compound (1), in the breast milk, and the daily dose of Compound (1) is adjusted to produce less than a maximum relative infant dose.

27. The method according to claim 26, wherein the maximum relative infant dose is at most about 0.5% of the daily dose administered to the subject.

28. The method according to claim 27, wherein the maximum relative infant dose is at most about 0.4% of the daily dose administered to the subject.

29. The method according to claim 28, wherein the maximum relative infant dose is at most about 0.357% of the daily dose administered to the subject.

30. The method according to any one of claims 1-29, wherein the child is monitored for abnormal behavior.

31. The method according to claim 30, wherein the abnormal behavior is selected from the group consisting of agitation, irritability, lethargy, oversleeping, poor feeding, and poor weight gain.

32. The method according to any one of claims 26-31, wherein the daily dose administered to the subject is decreased by 10 mg if the relative infant dose is above the maximum relative infant dose, or if the child shows abnormal behavior.

33. A method of treating major depressive disorder (MDD) in a human female subject, the method comprising administering to the subject a therapeutically effective amount of Compound (1): 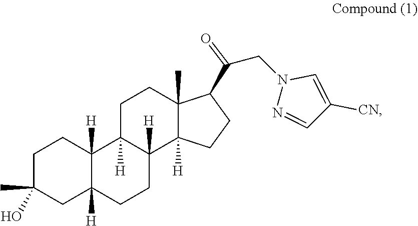
for a treatment period, wherein the subject breastfeeds a child during the treatment period.

34. A method of treating major depressive disorder (MDD) in a human female subject, the method comprising administering to the subject a therapeutically effective amount a pharmaceutically acceptable salt of Compound (1): 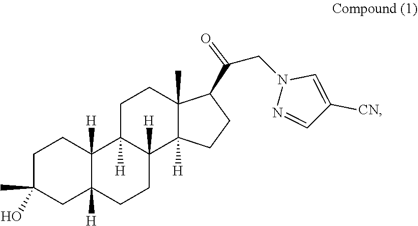
for a treatment period, wherein the subject breastfeeds a child during the treatment period.

35. A method of treating major depressive disorder (MDD) with elevated anxiety in a human female subject, the method comprising administering to the subject a therapeutically effective amount of Compound (1): 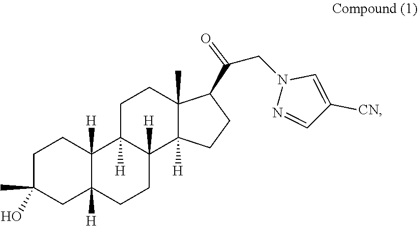
for a treatment period, wherein the subject breastfeeds a child during the treatment period.

36. A method of treating major depressive disorder (MDD) with elevated anxiety in a human female subject, the method comprising administering to the subject a therapeutically effective amount a pharmaceutically acceptable salt of Compound (1): 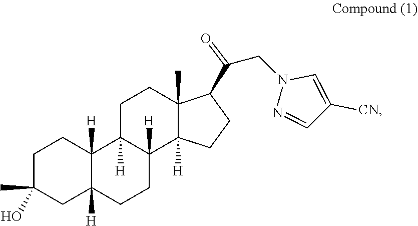
for a treatment period, wherein the subject breastfeeds a child during the treatment period.

37. The method according to any one of claims 33-36, wherein the subject breastfeeds the child at least 3 times per day.

38. The method according to any one of claims 33-36, wherein the treatment period is about 2 weeks or about 14 days.

39. The method according to any one of claims 33-36, wherein Compound (1), or the pharmaceutically acceptable salt of Compound (1), is administered once a day for about 14 days.

40. The method according to claim 33 or claim 35, wherein Compound (1) is administered at a dose of about 20 mg to about 55 mg.

41. The method according to claim 33 or claim 35, wherein Compound (1) is administered at a dose of about 50 mg.

42. The method according to claim 33 or claim 35, wherein Compound (1) is administered at a dose of about 40 mg.

43. The method according to claim 33 or claim 35, wherein Compound (1) is administered at a dose of about 30 mg.

44. The method according to claim 34 or claim 36, wherein the pharmaceutically acceptable salt of Compound (1) is administered at a dose equivalent to about 20 mg to about 55 mg of the free base compound.

45. The method according to claim 34 or claim 36, wherein the pharmaceutically acceptable salt of Compound (1) is administered at a dose equivalent to about 50 mg of the free base compound.

46. The method according to claim 34 or claim 36, wherein the pharmaceutically acceptable salt of Compound (1) is administered at a dose equivalent to about 40 mg of the free base compound.

47. The method according to claim 34 or claim 36, wherein the pharmaceutically acceptable salt of Compound (1) is administered at a dose equivalent to about 30 mg of the free base compound.

48. The method according to any one of claims 33-47, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasally, or transdermally.

49. The method according to claim 48, wherein Compound (1), or the pharmaceutically acceptable salt of Compound (1), is administered orally.

50. The method according to any one of claims 33-49, wherein Compound (1), or the pharmaceutically acceptable salt of Compound (1), is administered with food.

51. The method according to any one of claims 33-50, wherein Compound (1), or the pharmaceutically acceptable salt of Compound (1), is administered once a day at night.

52. The method according to claim 33 or claim 35, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

53. The method according to claim 33 or claim 35, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

54. The method according to claim 33 or claim 35, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

55. The method according to claim 33 or claim 35, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

56. The method according to any one of claims 33-55, wherein the subject is treatment naïve.

57. The method according to any one of claims 33-55, wherein the subject has been on a stable dose of an additional antidepressant for at least 30 days or for at least 60 days prior to the beginning of the treatment period.

58. The method according to any one of claims 33-57, wherein the breast milk of the subject is monitored to determine relative infant dose of Compound (1), or the pharmaceutically acceptable salt of Compound (1), in the breast milk, and the daily dose of Compound (1) is adjusted to produce less than a maximum relative infant dose.

59. The method according to claim 58, wherein the maximum relative infant dose is at most about 0.5% of the daily dose administered to the subject.

60. The method according to claim 59, wherein the maximum relative infant dose is at most about 0.4% of the daily dose administered to the subject.

61. The method according to claim 60, wherein the maximum relative infant dose is at most about 0.357% of the daily dose administered to the subject.

62. The method according to any one of claims 33-61, wherein the child is monitored for abnormal behavior.

63. The method according to claim 62, wherein the abnormal behavior is selected from the group consisting of agitation, irritability, lethargy, oversleeping, poor feeding, and poor weight gain.

64. The method according to any one of claims 58-63, wherein the daily dose administered to the subject is decreased by 10 mg if the relative infant dose is above the maximum relative infant dose, or if the child shows abnormal behavior.

65. The method according to any one of claims 1-64, further comprising administration of a second therapeutic agent.

66. The method according to any one of claims 1-65, wherein Compound (1), or the pharmaceutically acceptable salt of Compound (1), is re-administered to the subject in response to a recurrence of depression symptoms after completion of initial treatment period.

67. The method according to claim 66, wherein there is at least a 6 week interval between the last dose of initial treatment period and the first dose of the re-administration.

68. A method of treating postpartum depression (PPD) in a human female subject during the subject's postnatal period, the method comprising:
a) administering to the subject about 30 mg to about 50 mg of Compound (1) once a day for about 14 days: 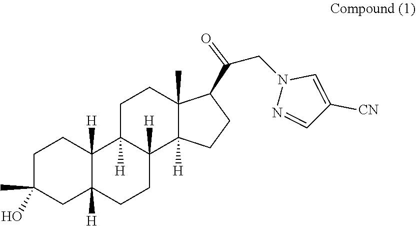
for a treatment period, wherein the subject breastfeeds a child during the treatment period;
b) comparing the relative infant dose of Compound (1) in the subject's breast milk with a predetermined maximum relative infant dose;
c) lowering the daily dose administered to the subject if the relative infant dose of Compound (1) in the subject's breast milk is above the predetermined maximum relative infant dose, or if the child is exhibiting abnormal behavior.

69. A method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days: 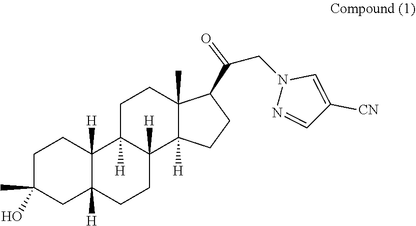
for a treatment period, wherein the subject breastfeeds a child during the treatment period;
b) comparing the relative infant dose of Compound (1) in the subject's breast milk with a predetermined maximum relative infant dose;
c) lowering the daily dose administered to the subject if the relative infant dose of Compound (1) in the subject's breast milk is above the predetermined maximum relative infant dose, or if the child is exhibiting abnormal behavior.

70. A method of treating major depressive disorder (MDD) in a human female subject, the method comprising:
a) administering to the subject about 30 mg to about 50 mg of Compound (1) once a day for about 14 days: 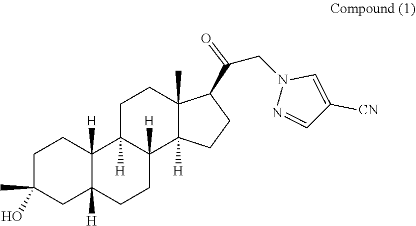
for a treatment period, wherein the subject breastfeeds a child during the treatment period;
b) comparing the relative infant dose of Compound (1) in the subject's breast milk with a predetermined maximum relative infant dose;
c) lowering the daily dose administered to the subject if the relative infant dose of Compound (1) in the subject's breast milk is above the predetermined maximum relative infant dose, or if the child is exhibiting abnormal behavior.

71. A method of treating major depressive disorder (MDD) in a human female, the method comprising:
a) administering to the subject a pharmaceutically acceptable salt of Compound (1) at a dose equivalent to about 30 mg to about 50 mg of the free base compound once a day for about 14 days: 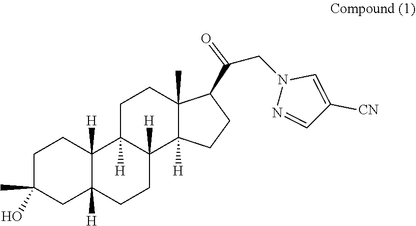
for a treatment period, wherein the subject breastfeeds a child during the treatment period;
b) comparing the relative infant dose of Compound (1) in the subject's breast milk with a predetermined maximum relative infant dose;
c) lowering the daily dose administered to the subject if the relative infant dose of Compound (1) in the subject's breast milk is above the predetermined maximum relative infant dose, or if the child is exhibiting abnormal behavior.

72. A method of treating postpartum depression (PPD) in a human female subject during the subject's postnatal period, the method comprising:
a) administering to the subject about 30 mg to about 50 mg of Compound (1) once a day for about 14 days: 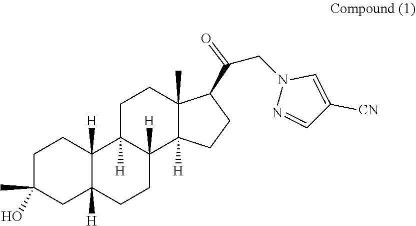
for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

73. A method of treating postpartum depression (PPD) in a human female subject during the subject's postnatal period, the method comprising:
a) administering to the subject a pharmaceutically acceptable salt of Compound (1) at a dose equivalent to about 30 mg to about 50 mg of the free base compound once a day for about 14 days: 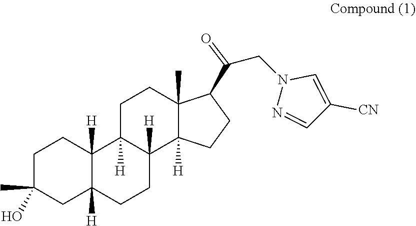
for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

74. A method of treating major depressive disorder (MDD) in a human female subject, the method comprising:
a) administering to the subject about 30 mg to about 50 mg of Compound (1) once a day for about 14 days: 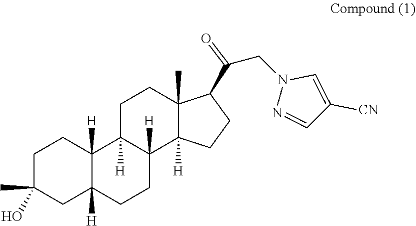
for a treatment period, wherein the subject breastfeeds a child during the treatment period;
b) collecting and testing a sample of the subject's breast milk to determine the relative infant dose of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

75. A method of treating major depressive disorder (MDD) in a human female, the method comprising:
a) administering to the subject a pharmaceutically acceptable salt of Compound (1) at a dose equivalent to about 30 mg to about 50 mg of the free base compound once a day for about 14 days: 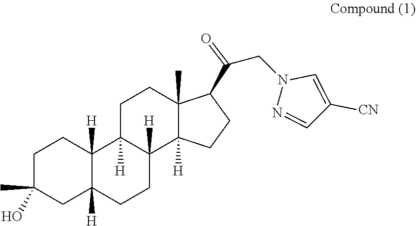
for a treatment period, wherein the subject breastfeeds a child during the treatment period;
b) collecting and testing a sample of the subject's breast milk to determine the relative infant dose of the pharmaceutically acceptable salt of Compound (1) in the breast milk;
c) comparing the relative infant dose determined in step b) with a predetermined maximum relative infant dose;
d) monitoring the child for abnormal behavior;
e) lowering the daily dose administered to the subject if the relative infant dose determined in step b) is above the predetermined maximum relative infant dose, or if the child is exhibiting abnormal behavior; and
f) repeating steps a)-e) each day for the duration of the treatment period.

76. The method according to any one of claims 68-69 or 72-73, wherein the PPD is PPD with elevated anxiety.

77. The method according to any one of claims 70-71 or 74-75, wherein the MDD is MDD with elevated anxiety

78. The method according to any one of claims 68-77, wherein Compound (1) is administered at a dose of about 50 mg or the pharmaceutically acceptable salt of Compound (1) is administered at a dose equivalent to about 50 mg of the free base compound.

79. The method according to any one of claims 68-77, wherein Compound (1) is administered at a dose of about 40 mg or the pharmaceutically acceptable salt of Compound (1) is administered at a dose equivalent to about 40 mg of the free base compound.

80. The method according to any one of claims 68-77, wherein Compound (1) is administered at a dose of about 30 mg or the pharmaceutically acceptable salt of Compound (1) is administered at a dose equivalent to about 30 mg of the free base compound.

81. The method according to any one of claims 68-80, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasally, or transdermally.

82. The method according to claim 81, wherein Compound (1), or the pharmaceutically acceptable salt of Compound (1), is administered orally.

83. The method according to any one of claims 68-82, wherein Compound (1), or the pharmaceutically acceptable salt of Compound (1), is administered with food.

84. The method of any one of claim 68-83, wherein Compound (1), or the pharmaceutically acceptable salt of Compound (1), is administered once a day at night.

85. The method according to any one of claims 68-84, wherein the subject is treatment naïve.

86. The method according to any one of claims 68-84, wherein the subject has been on a stable dose of an additional antidepressant for at least 30 days or for at least 60 days prior to the beginning of the treatment period.

87. The method according to any one of claims 68-86, further comprising administration of a second therapeutic agent.

88. The method according to any one of claims 68-87, wherein Compound (1), or the pharmaceutically acceptable salt of Compound (1), is re-administered to the subject in response to a recurrence of depression symptoms after completion of initial treatment period.

89. The method according to claim 88, wherein there is at least a 6 week interval between the last dose of the initial treatment period and the first dose of the re-administration.

90. The method according to any one of claims 68-89, wherein the predetermined maximum relative infant dose is at least about 0.5% of the daily dose administered to the subject.

91. The method according to claim 90, wherein the maximum relative infant dose is at least about 0.4% of the daily dose administered to the subject.

92. The method according to claim 91, wherein the maximum relative infant dose is at least about 0.357% of the daily dose administered to the subject.

93. The method according to any one of claims 68-89, wherein the abnormal behavior for the infant is selected from the group consisting of agitation, irritability, lethargy, oversleeping, poor feeding, and poor weight gain.
